(19) United States
(12) Patent Application Publication
(10) Pub. No.: US 2019/0307690 A1
(43) Pub. Date: Oct. 10, 2019

(54) Liposomal Delivery Systems for Oxaliplatin and in Dual Drug Delivery in Combination with Chemo-sensitizing and Chemo-therapeutic agents

(71) Applicant: THE AMERICAN UNIVERSITY IN CAIRO, New York, NY (US)

(72) Inventors: Tamer Shoeib, New Cairo (EG); Ayat Zein-elabedeen Mohamed, Nasr City (EG)

(21) Appl. No.: 16/098,509

(22) PCT Filed: May 2, 2017
(86) PCT No.: PCT/US17/30526

§ 371 Date: Nov. 2, 2018

Related U.S. Application Data
(60) Provisional application No. 62/330,955, filed on May 3, 2016.

(51) Int. Cl. A61K 9/127; A61K 9/00; A61K 31/555; A61K 33/243

(52) U.S. Cl. CPC A61K 9/1271; A61K 33/243; A61K 31/555; A61K 9/0019

(57) ABSTRACT

A liposomal delivery composition to be administered through intravenous injection is provided for the treatment of cancer. The delivery composition has a liposome composition with therein encapsulated a first cancer drug (e.g. oxaliplatin) and a second cancer drug (e.g. ascorbic acid or satraplatin). The liposomal delivery composition has negative surface potentials resulting in an encapsulation efficiency for e.g. of oxaliplatin of about 20-25%. The liposomal delivery composition has a particle size of less than 200 nm. The liposomal delivery offers protection of the drug cargo, which reduces their non-intentional and non-pharmacological interactions thus reducing side effects and increases efficacy. Increased efficacy reduces the amount of drug given to a patient, which would reduce healthcare cost. The combinatory approach of two different drug cargos within the same liposome delivery composition allows for the synergistic action of these drugs.

### FIELD OF THE INVENTION

The invention relates to cancer drug delivery systems.

### BACKGROUND OF THE INVENTION

Cancer is reported by the World Health Organization (WHO) to be one of the rapidly growing leading causes of death worldwide, with an estimate of 8.2 million cancer-related deaths, and around 14.1 million new cancer cases in 2012, compared with 12.7 million new case in 2008. Progression of cancer can be controlled using several interventions such as, surgery, radiation, immunotherapy, suicide gene therapy, and chemotherapy; where most of these interventions induce their anticancer effect by inhibiting cancer cell proliferation, that might lead to senescence or activation of cell death pathways through apoptosis, necrosis, and mitotic catastrophe in tumor cells. Chemotherapeutic agents are used widely post-surgery and radiotherapy as an adjuvant therapy to eradicate residual cancer cells, also used as a palliative treatment where it aids in reducing tumor size, or for complete cure of cancer. Drug delivery constitutes a major segment in chemotherapeutics including liposomes as a drug delivery system enabling the encapsulation of drugs.

Liposomes have been recognized as an efficient means for drug delivery. The present invention advances the art by providing liposomal delivery systems for dual drug delivery for cancer therapy.

### SUMMARY OF THE INVENTION

A liposomal delivery composition to be administered through intravenous injection is provided for the treatment of cancer. The delivery composition has a liposome composition which is composed of: distearoyl phosphatiylcholine (DSPC), distearoyl phosphoethanolamine (DSPE), distearoyl phosphatidylethanolamine-polyethylene glycol (DSPE-PEG), and cholesterol. The molar ratio for DSPC in the composition ranges from 30-50%. The molar ratio for DSPE in the composition ranges from 3-5%. The molar ratio for DSPE-PEG in the composition ranges from 5-40%. The molar ratio for cholesterol in the composition ranges from 25-40%. In one example, the DSPE-PEG is phosphatidylethanolamine-polyethylene glycol 2000 (DSPE-PEG 2000).

A first cancer drug and a second cancer drug are encapsulated by the liposome delivery composition such that the first cancer drug is different from the second cancer drug. In one embodiment, the first cancer drug is oxaliplatin and the second cancer drug is ascorbic acid. In another embodiment, the first cancer drug is oxaliplatin and the second cancer drug is satraplatin. In more general terms, the second cancer drug can either be hydrophilic or hydrophobic.

When the first drug is oxaplatin and the second drug is ascorbic acid, a molar ratio is defined such that the ascorbic acid is a fraction of about 0.01 to 0.05 higher than that of the oxaplatin. Specifically, oxaplatin:ascorbic acid=1.00:1.02. When the first drug is oxaplatin and the second drug is satraplatin, a molar ratio is defined such that the satraplatin is about 5 times higher than than that of oxaplatin. Specifically, oxaplatin:satraplatin=1.00:4.90.

The liposomal delivery composition has negative surface potentials resulting in an encapsulation efficiency for e.g. of oxaliplatin of about 20-25%. The liposomal delivery composition has a particle size of less than 200 nm.

The liposomal delivery offers protection of the drug cargo, which reduces their non-intentional and non-pharmacological interactions thus reducing side effects and increases efficacy. Increased efficacy reduces the amount of drug given to a patient, which would reduce healthcare cost. The combinatory approach of two different drug cargos within the same liposome delivery composition allows for the synergistic action of these drugs.

### BRIEF DESCRIPTION OF THE DRAWINGS

FIGS. 1A-C show according to an exemplary embodiment of the invention a TEM images indicating the formation of small unilamellar liposomal vesicle with PEG coat on the surface. (FIG. 1A) LP-Ox (FIG. 1B) LP-Ox-AA (FIG. 1C) LP-Ox-Stp.

FIGS. 2A-C show according to an exemplary embodiment of the invention a FT-IR spectra for the prepared liposomal formulations. (FIG. 2A) LP-Ox, (FIG. 2B) LP-Ox-AA, (FIG. 2C) LP-Ox-Stp.

FIGS. 3A-B show according to an exemplary embodiment of the invention Cell means plot for (FIG. 3A) Size, and (FIG. 3B) potential of samples stored for a 6 month duration at 4° C.

FIGS. 4A-E show according to an exemplary embodiment of the invention the drug release profiles for the prepare formulations. (FIG. 4A) Free Oxaliplatin release profile, (FIG. 4B) Lipoxal release profile of oxaliplatin, (FIG. 4C) LP-Ox release profile of oxaliplatin, (FIG. 4D) LP-Ox-AA release profile of oxaliplatin, (FIG. 4E) LP-OX-Stp release profile of oxaliplatin and satraplatin.

FIGS. 5A-B show according to an exemplary embodiment of the invention a comparative study for the oxaliplatin release profile for prepared liposomal formulation against (FIG. 5A) Free oxaliplatin, and (FIG. 5B) Lipoxal.

FIG. 6 shows according to an exemplary embodiment of the invention a comparative study for the oxaliplatin release profile for single drug loaded liposomal formulation LP-Ox against Free oxaliplatin, and oxaliplatin spiked void liposome LP-void+Oxpt.

FIGS. 7A-B show according to an exemplary embodiment of the invention a comparative study for dual drug loaded liposomal formulation LP-Ox-Stp (FIG. 7A) the satraplatin release profile against single drug loaded liposomal formulation LP-Stp, and oxaliplatin spiked liposomal formulation LP-Stp+Oxpt; (FIG. 7B) the oxaliplatin release profile against single drug loaded liposomal formulation LP-Ox, and oxaliplatin spiked liposomal formulation LP-Stp+Oxpt.

FIGS. 8A-C show according to an exemplary embodiment of the invention effects of prepared liposomal formulations on cell viability of (FIG. 8A) MCF-7, (FIG. 8B) HepG2, and (FIG. 8C) BHK-21 cell lines relative to free oxaliplatin drug solution, the results are expressed as a percent of the control.

FIG. 9 shows according to an exemplary embodiment of the invention a comparative IC50 for the prepared formulations in different cell lines versus free oxaliplatin and Lipoxal. Data are means±SD, n=2. *: P<0.05 difference from Free oxaliplatin. **: P<0.01 difference from Free oxaliplatin.

FIG. 10 shows according to an exemplary embodiment of the invention immunofluorescence images for studying oxaliplatin induced DNA damage.

FIGS. 11A-B show according to an exemplary embodiment of the invention magnitude of oxaliplatin and liposomal formulations induced DNA damage in MCF-7 cell line. (FIG. 11A) γ-H2AX foci analysis treated with 2 uM oxaliplatin, (FIG. 11B) % cells with γ-H2AX foci pan-nuclear staining.

FIGS. 12A-C show according to an exemplary embodiment of the invention calibration curves for oxaliplatin (FIG. 12A), ascorbic acid (FIG. 12B), and satraplatin (FIG. 12C).

### BRIEF DESCRIPTION OF THE TABLES

Tables can be found towards the end of the specification.

Table 1 Structures, and relative charges of lipid components.

Table 2 Mole Ratio of lipids and oxaliplatin used to prepare DSPG containing liposomes.

Table 3 Mole Ratio of lipids and drugs used to prepare dual drug loaded liposomes.

Table 4 Lipophilicity of drug used in liposome preparation.

Table 5 The effect of single drug loading on size, Polymer dispersity index (PDI), and potential, measures of stability.

Table 6 The effect of DSPG incorporation on liposomal formulation.

Table 7 Influence of dual drug loading on liposomal formulation and Lipoxal characterization results.

Table 8 Liposome characterization results over 6 month storage duration.

Table 9 Difference and Similarity factors for comparative study.

Table 10 Coefficient of determination, and drug release rates obtained from different mathematical model fitting of release data.

Table 11 In-vitro cytotoxicity of prepared liposomal formulations.

### LIST OF ABBREVIATIONS

ATM ataxia telangiectasia mutated
ATR ataxia telangiectasia mutated and Rad3-related
CTR1 copper transporter 1
DACH 1,2-diaminocyclohexane group
DCM dichloromethane
DMEM Dulbecco's Modified Eagle Medium
DMSO Dimethyl sulfoxide
DNA Deoxyribonucleic acid
DPPC dipalmitoyl phosphatidylcholine
DPPE dipalmitoyl phosphatidylethanolamine
DPPG dipalmitoyl phosphatidylglycerol
DSB double stranded DNA breaks
DSPC Distearoyl phosphatiylcholine
DSPE Distearoyl phosphoethanolamine
DSPE-PEG 2000 Distearoyl phosphatidylethanolamine-polyethylene glycol 2000
DSPG Distearoyl phosphatidylglycerol
EGCG Epigallocatechin-3-gallate
EPR Enhanced permeability and retention
FBS Fetal bovine serum
FDA US Food and Drug Administration
FTIR Fourier transform infrared spectroscopy
OSH Reduced glutathione
HII Hexagonal phase
IC50 The half maximal inhibitory concentration
LC Lipid concentration
LUV Large unilamellar vesicles
MAPK mitogen-activated protein kinase
MDR Multi-drug resistance
MMR Mismatch repair
MPS Mononuclear phagocytic system
MTD Maximum tolerated dose
MVV Multivesicular vesicles
MWCO Molecular weight cut off
NER Nucleotide excision repair
OCT Organic cation transporters
Oxpt Oxaliplatin
PBS Phosphate buffer saline
PC Phosphatidylcholine
PDI Polydispersity index
PE Phosphatidylethanolamine
PEG Polyethylene glycol
PG Phosphatidylglycerol
pKa acid dissociation constant
PP Pellet permeabilization
PS Phosphatidylserine
R² Coefficient of determination
RBCs Rod blood cells
RES Reticuloendothelial system
SD Standard deviation
SDS Sodium dodecyl sulphate
SE Standard Error
SSB Single stranded DNA breaks
TEM Transmission electron microscopy
UC Ultracentrifugation
WHO World health organization
ζ potential zeta potential DETAILED DESCRIPTION

This invention provides technology, which incorporates the use of liposomes as a dual delivery system for oxaliplatin and satraplatin as well as for oxaliplatin and ascorbic acid aimed at the synergistic effect of these combinations and reducing their toxicity profiles.

Materials and Methods

Chemicals and reagents

| | |
|---|---|
| L-(+)-Ascorbic acid | NenTech Ltd., Northants, UK |
| Sucrose 98% | Aldrich chemical company Inc., |
| | Wisconsin, USA |
| Dimethylthiazol diphenyl tetrazolium | Serva, Germany |
| bromide | |
| Sodium dodecylsulphate (SDS) | Sigma-Aldrich, USA |
| Dichloromethane, HPLC grade, | Scharlau, Barcelona, Spain |
| stabilized with ethanol | |
| J T Baker ® Acetonitrile HPLC Far | Avantor performance materials, |
| UV/Gradient Grade | Pennsylvania, USA |
| J T Baker ® Methanol (ultra) | Avantor performance materials, |
| Gradient HPLC grade | Pennsylvania, USA |
| Dimethyl sulfoxide (DMSO) | Sigma-Aldrich, USA |
| Biowhittaker ® Phosphate Buffered | Lonza, Basel, Switzerland |
| Saline 0.0067M (PO₄) without | |
| Ca and Mg | |
| Dulbecco's Modified Eagle Medium | Lonza, Basel, Switzerland |
| (DMEM) supplemented with 10% Fetal | |
| bovine serum (FBS) and 5% Penicillin- | |
| Streptomycin mixture | |
| Milli-Q ultrapure water | Obtained by Millipore system |

Platinum Complexes

| | |
|---|---|
| Oxaliplatin | Sanofi-Synthelabo Limited and Shandong Boyuan |
| | Pharmaceutical Co. Ltd. |
| Satraplatin | Sanofi-Synthelabo Limited and Shandong Boyuan |
| | Pharmaceutical Co. Ltd., |
| Lipoxal ™ | Regulon Inc., Attica, Greece |

Lipid

| | |
|---|---|
| 1,2-Distearoyl-sn-glycero-3-phosphocholine | Corden Pharma, |
| (DSPC) | Plankstadt, Germany |
| 1,2-Distearoyl-sn-glycer-3-phosphoethanolamine | Corden Pharma, |
| (DSPE) | Plankstadt, Germany |
| 1,2-Distearoyl-sn-glycero-3-phospho-rac-glycerol, | Lipoid, Steinhausen, |
| sodium salt, (DSPG-Na) | Switzerland |
| N-(Carbonyl-methoxypolyethyleneglycol 2000)- | Corden Pharma, |
| 1,2-distearoyl-sn-glycero-3-phosphoethanolamine | Plankstadt, Germany |
| sodium salt (MPEG-2000-DSPE) | |
| Cholesterol | Corden Pharma, |
| | Plankstadt, Germany |

An overview of the lipid structures in shown in Table 2.

Solutions

Oxaliplatin solution at 7.55 mM concentration, a 7.52 mM Ascorbic acid, and 19.28 mM Sodium dodecylsulphate (SDS) solutions were prepared in ultrapure water.

Cell Lines

The human mammary gland adenocarcinoma cell line MCF-7, human liver hepatocellular carcinoma HepG2, and human kidney normal cells, BHK-21 (kindly provided by Dr. Sameh Saad Ali, Zewail City of Science and Technology, Egypt) were used in the study. MCF-7 is reported as a relatively resistant cell line to cisplatin compared to other breast cancer cell lines, however it does not develop resistance to oxaliplatin (du Plessis-Stoman et al., Combination Treatment With Oxaliplatin and Mangiferin Causes, Afr J Tradit Complement Altern Med, vol. 8, no. 2, pp. 177-184, 2011; and Yde et al., Enhancing cisplatin sensitivity in MCF-7 human breast cancer cells by down-regulation of Bcl-2 and cyclin Dl., Int. J. Oncol., vol. 29, no. 17, pp. 1397-1404, 2006). The cell lines were from organism: Homo sapiens, human (Tissue: mammary gland, breast; derived from metastatic site: pleural effusion, Disease: adenocarcinoma).

Consumable materials

| | |
|---|---|
| Pippette tips | Socorex, Ecublens, Switzerland |
| Nanocep ® MF centrifugal devices | Pall Life Sciences, USA |
| with GHP membrane | |
| Visking ® dialysis tubing (MWCO | SERVA, Germany |
| 12-14 KDa) | |
| Eppendorf microcentrifuge tubes - | Fisher scientific, UK |
| Safe lock (2 ml) | |
| Falcon ™ tubes - Conical | Fisher scientific, UK |
| centrifuge (15 ml) | |
| Parafilm M ® | Parafilm - Bemis, USA |
| Clear glass screw thread vials | Thermo Fisher Scientitfic Inc., USA |
| Tissue culture plates, 96 wells | Greiner Labortechnik, Germany |

Equipment

| | |
|---|---|
| Rotavapor ® | BÜCHI, Flawil, Switzerland |
| Cooling centrifuge | HERMLE LABORTECHNIK, |
| | Wehingen, Germany |
| Grant Bio PV-1 Vortex mixer | Grant instruments (Cambridge) |
| | Ltd., UK |
| Analytical balance | Mettler Toledo, USA |
| Hot plate | VWR, USA |
| 10, 200, 1000 μl Micropipettes | Eppendorf, Germany |
| Shaking water bath | Grant instruments (Cambridge) |
| | Ltd., UK |
| Microplate reader | FLUOstar OPTIMA (BMG |
| | LabTech, Germany) |
| Countess ® Automated cell counter, | Thermo Fisher Scientitfic Inc., |
| Invitrogen | USA |

Zetasizer

| | |
|---|---|
| Zetasizer | Malvern Instruments Ltd., UK |
| Disposable capillary cells (DTS1070) | Malvern Instruments Ltd., UK |

Ultra High Performance Liquid Chromatography

| | |
|---|---|
| Ultimate UHPLC dionex | Thermo Fisher Scientitfic Inc., USA |
| BDS Hypersil C18 column | Thermo Fisher Scientitfic Inc., USA |
| (250 × 4.6 mm) | |
| Photodiode array detector | Thermo Fisher Scientitfic Inc., USA |

Transmission electron microscopy

| | |
|---|---|
| Transmission electron microscope | JEOL, Massachusetts, USA |
| Carbon coated copper grid | Polysciences Inc., Germany |

Membrane extrusion

| | |
|---|---|
| Avanti ® Mini-extruder | Avanti polar lipids Inc., USA |
| Extruder set with heating block | Avanti polar lipids Inc., USA |
| Polycarbonate membrane 0.1 μm | Avanti polar lipids Inc., USA |
| Filter supports | Avanti polar lipids Inc., USA |
| Gas tight syringe | Avanti polar lipids Inc., USA |

Software

| | |
|---|---|
| Chromeleon ™ Software | Thermo Fisher Scientitfic Inc., USA |
| Malvern | Malvern Instruments Ltd., UK |
| Controller for JEM-2100/HT | JEOL, Massachusetts, USA |

Preparation of Liposomes

Stealth liposomes were prepared using the thin-film hydration method followed by membrane extrusion to control the particle diameter as previously described by Nallamo (Nallamothu et al., Targeted Liposome Delivery System for Combretastatin A4: Formulation Optimization Through Drug Loading and In Vitro Release Studies, PDA J Pharm Sci Technol. 2006 May-June; 60(3): 144-55). For the preparation of oxaliplatin loaded liposomes, lipids were dissolved in a 250 ml round bottomed flask containing a sufficient amount of dichloromethane forming a lipid mixture. This was followed by the removal of the organic phase by rotary evaporation under reduced pressure at 60° C., a temperature equivalent to the lipids transition temperature (Tm), to obtain a continuous thin film of lipids on the flask wall. The dry thin film was subsequently hydrated with 3 ml of 7.55 mM oxaliplatin solution, and was allowed to resume rotation in a rotary evaporator under normal pressure at 60° C. for 2 hours. Finally, membrane extrusion was performed using 100 nm polycarbonate membranes at 60° C. The prepared liposomal formulations were stored at 4° C. In the preparation of “void” liposomes or unloaded liposomes; ultrapure water was added instead of the drug solution during the hydration phase.

Since the composition of the lipid membrane tends to influence the characteristics of the prepared liposomes, part of the invention has focused on the comparison of different lipid compositions and their influence on the liposome characterization. DSPG, a negatively charged lipid, was used in the liposome formulation to displace 5% of the total mole percent of each of the other lipid components used being DSPE, DSPC, and Cholesterol, as stated in Table 2.

For the preparation of dual-drug loaded liposomes, the hydrophilic/hydrophobic nature of the added drug influence the stage of its addition during liposome preparation. As stated in Table 4, hydrophobic drugs such as satraplatin are added to the lipid mixture prior to the formation of the thin film, whereas hydrophilic drugs such as ascorbic acid are added to the hydration solution. The ratio of the additional drug used is stated in Table 3.

The dual drug loaded liposome containing oxaliplatin and ascorbic acid is encoded as LP-Ox-AA, and the liposomal formulation loaded with oxaliplatin and satraplatin is encoded as LP-Ox-Stp. In addition, a liposome formulation was prepared loaded only with satraplatin (LP-Stp) to evaluate the effect of loading a hydrophobic drug in the liposome lipid bilayer.

Liposome Characterization

Particle Size, Polydispersity Index, and Zetapotential

The particle size, polydispersity index (PDI), and Zeta potential (ζ potential) of liposomes were analyzed by Dynamic light scattering technique using a Zetasizer Nano Series (Malvern Instruments, UK). To ensure a convenient scattered intensity on the detector, formulations were diluted 1:50 (v/v) in ultrapure water prior to its measurement at 25° C.

Encapsulation Efficiency

Two different techniques were used determine the encapsulation efficiencies for loaded drug within the liposomal delivery system.

Liposomese Parathion Procedure

i) Ultracentrifugation (UC)

Ultracentrifugation was used to separate liposomes from the bulk solution, and determine the amount of drug encapsulated in the recovered liposomes. Briefly, 0.5 ml of 1:2 (v/v) diluted liposome preparation is added to the Nanosep centrifugal device, and was centrifuged at 7000 rpm at 4° C. for 1 h. This was followed by the analysis of the filtrate using HPLC. The Encapsulation efficiency expressed in percentage (%) was calculated using the following equations.

Total amount of drug encapsulated in liposomes=(Total amount of drug added−Un-entrapped drug added fraction).

Encapsulation efficiency %(EE %)=Total amount of drug in liposomes/Total amount of drug added)*100%

ii) Pellet Permeabilization (PP)

A total of 400 μl of 1:2 (v/v) diluted liposome preparation was centrifuged at 16500 rpm at 4° C. for 3 h. This was followed by collecting both the supernatant and the pellet, the pellet was diluted in 1:4 (v/v) 19.28 mM SDS in order to permeate the liposome entrapped drugs; then both the supernatant and the permeabilized pellets were analyzed using HPLC. The Encapsulation efficiency expressed in percentage (%) was calculated using the following equations.

Total amount of drug=(Total amount of drug encapsulated in pellet+Total amount of drug un-encapsulated in supernatant)

Encapsulation efficiency %(EE %)=(Total amount of drug encapsulated in pellet/Total amount of drug)*100%

Quantification Method

The liposome un-entrapped fraction of drugs were quantified using UHPLC, with a photodiode array detector and a BDS Hypersil C18 reverse-phase column (250 mm×4.6 mm, 5 mm). Two methods were followed.

i) Oxaliplatin Quantification

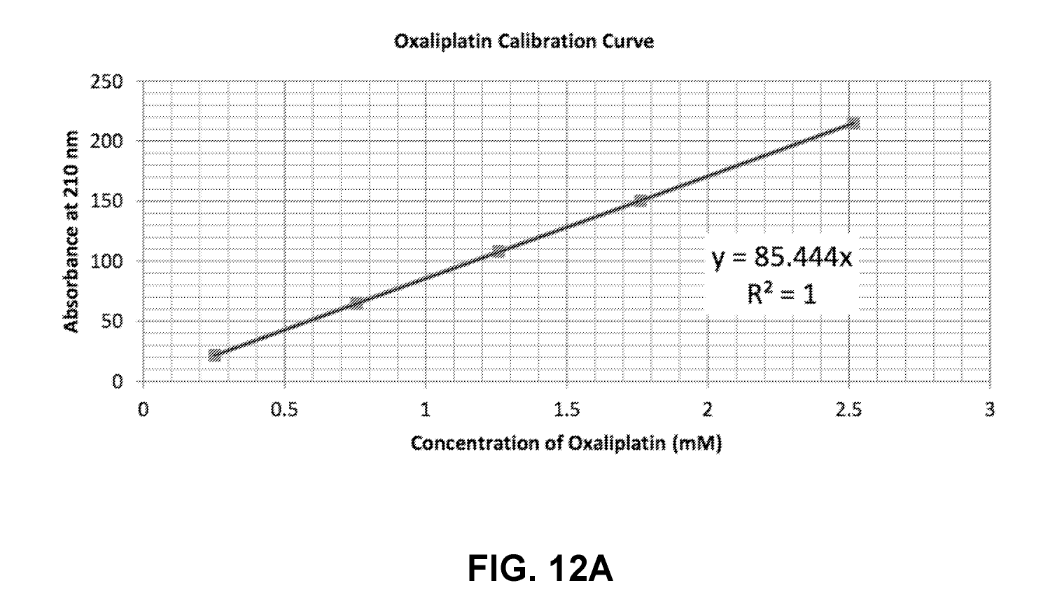

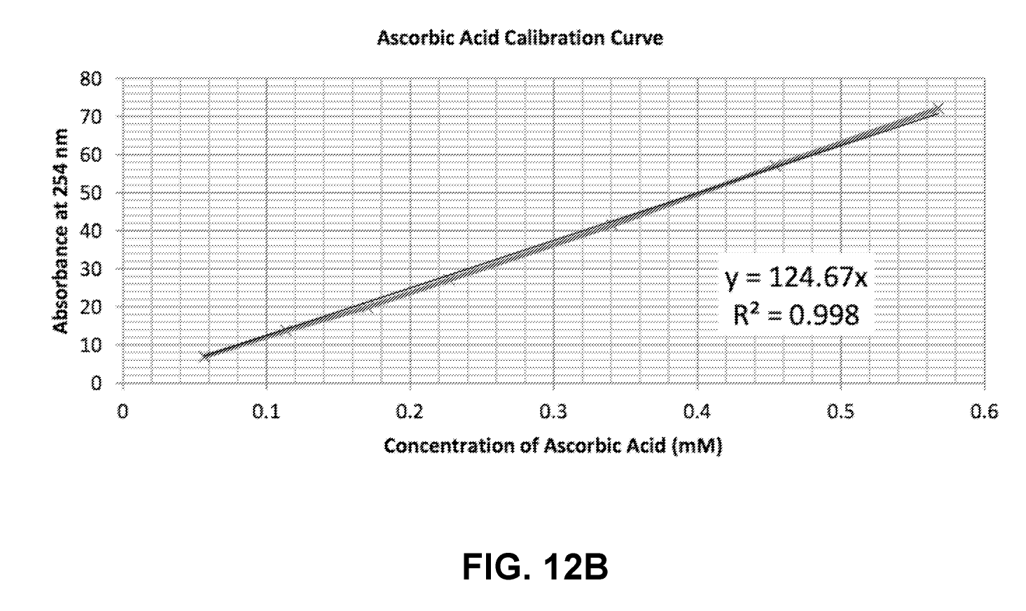

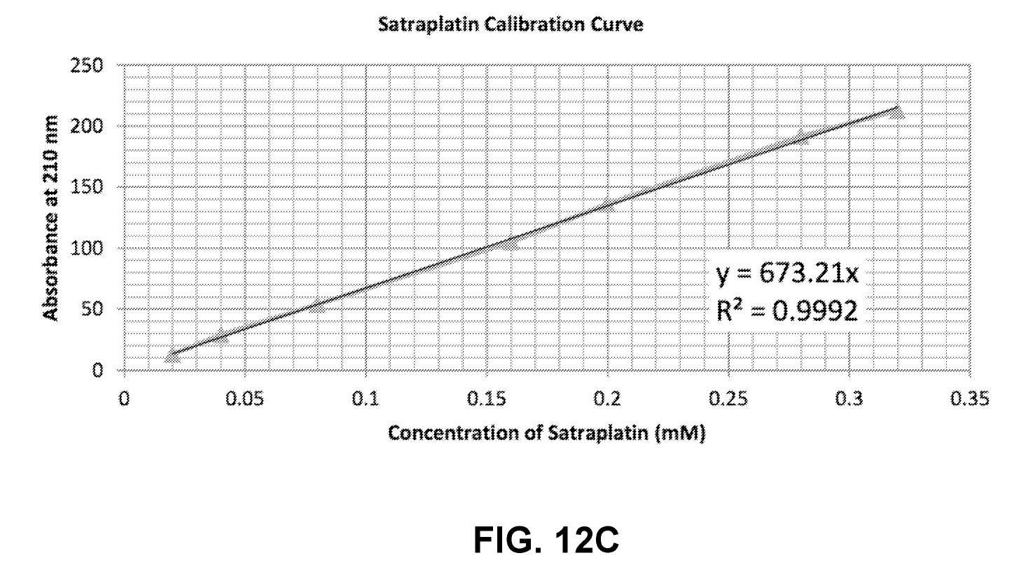

The mobile phase consisted of deionized water and acetonitrile (99:1) (v/v) at a flow rate of 1.2 ml/min, with the column temperature maintained at 40° C. The injection volume was 20 μL, and the effluent monitored at 210 nm. The sample oxaliplatin concentration was determined using the constructed calibration curve (FIGS. 12A-C).

ii) Satraplatin and Ascorbic Acid Quantification

The mobile phase was composed of deionized water and acetonitrile (50:50) (v/v) at a flow rate of 1 ml/min, with the column temperature maintained at 40° C. The injection volume was 20 μL, and the effluent was simultaneously monitored at 210 nm for detection of satraplatin, and at 254 nm for detection of ascorbic acid. The sample satraplatin and ascorbic acid concentration was determined using their respective constructed calibration curves (FIGS. 12A-C).

Transmission Electron Microscopy (TEM)

The prepared stealth liposomes were analyzed by TEM. The measurements were carried out by means of a JEOL-JEM 2100 electron microscope operating at 160 kV. Fifty microliter of the sample was deposited over a carbon-coated copper grid with 200 mesh and dried. The sample was then negatively stained with 2% aqueous phosphotungstic acid and dried. The sample was then visualized and photographed.

In-Vitro Drug Release Analysis

The drug release testing was conducted according to a described method (Shazly et al., Comparison of dialysis and dispersion methods for in vitro release determination of drugs from multilamellar liposomes, Dissolution Technol., vol. 15, no. 2, pp. 7-10, 2008). This was done for LP-Ox, LP-Ox-AA, LP-Ox-Stp, free oxaliplatin drug solution, Lipoxal (a commercial liposomal formulation of oxaliplatin), and finally LP-void as well as LP-Stp each spiked with an equivalent concentration of oxaliplatin. A volume of 0.5 ml of liposomal preparation was placed in a dialysis tubing (3.8 cm in length). Both ends were tied. The dialysis bag was suspended in 25 ml PBS at pH 7.4 and maintained at 37±0.5° C. (Shazly et al., 2008). The dispersion was rotated at 200 strokes/minute in a water bath shaker (Shazly et al., 2008). At predetermined time intervals of 0.25, 0.5, 1, 1.5, 2, 4, 6, 8, 12, 24, 48, and 72 h; 1 ml aliquots were sampled and replaced with 1 ml fresh pH 7.4 PBS, which was maintained at the same temperature as the samples being 37±0.5° C. Drug concentrations were determined using HPLC.

Release Kinetics

Two methods were used to investigate the kinetics of drug release profile from the prepared liposomal formulations.

Model Dependent Methods

This method involved the fitting of the release data to one of the following seven release kinetic models.

i) Zero-Order

Qₜ=Q₀+K₀t

Where, Qₜ is the amount of drug released in time t, Q₀ is the initial amount of drug in the solution and K₀ is the zero order release constant expressed in units of concentration/time. To study the release kinetics, data obtained from in-vitro drug release studies were plotted as cumulative amount of drug released versus time.

ii) First-Order

log Cₜ=log C₀−K₁t/2.303

Where, Cₜ is the concentration of drug released in time t, C₀ is the initial concentration of drug, K₁ is the first order rate constant. The data obtained were plotted as log cumulative percentage of drug remaining versus time which would yield a straight line with a slope of −K/2.303.

iii) Higuchi

Q=K_{H}×t^{1/2}

Where, Q is the amount of drug released in time t, K_{H} is the Higuchi dissolution constant. The data obtained were plotted as cumulative percentage of drug released versus square root of time.

iv) Hixson-Crowell

Q₀^{1/3}−Qₜ^{1/3}=K_{C}t

Where, Qₜ is the amount of drug remaining in time t, Q₀ is the initial amount of the drug in liposome and K_{C} is the rate constant for Hixson-Crowell rate equation. The data obtained were plotted as Cube root of cumulative percentage of drug remaining versus time.

v) Korsmeyer-Peppas

Mₜ/M_{∞}=K_{P}tⁿ

Where Mₜ/M_{∞} is a fraction of drug released at time t, K_{P} is the release rate constant and n is the release exponent. The data obtained were plotted as log (cumulative percentage of drug released) versus log (time).

vi) Baker Lonsdale

$${f}_{1} = \frac{3}{2} [ 1 - {( 1 - \frac{{M}_{t}}{{M}_{∞}} )}^{2 / 3} ] \frac{{M}_{t}}{{M}_{∞}} = {K}_{B ( t )}$$

Where [Mₜ/M_{∞}] is a fraction of drug released at time t, K_{B} is the release rate constant. To study the release kinetics, data obtained from in-vitro drug release were plotted as [d(Mₜ/M_{∞})/dt] against the root of time inverse on x-axis.

vii) Michaelis-Menten (Hyperbola)

dC/dt=VₘC/(Kₘ+C)

Where Vₘ is the maximum release rate, Kₘ is the Michaelis-Menten constant, C is the amount of drug released, and t is time. The data obtained were plotted as drug release % versus time. Michaelis-menten was previously reported in describing the release kinetics of Ketorolac from silica nanoparticles (Lopez Goerneet al., Obtaining of sol-gel ketorolac-silica nanoparticles: Characterization and drug release kinetics, J. Nanomater., vol. 2013, 2013).

Model Independent Method

This method utilizes the difference factor (f₁), similarity factor (f₂) to compare the release profiles of different formulations by measuring the percent difference and the percent similarity respectively.

$${f}_{1} = \frac{{\sum }_{t = 1}^{n} \vert {R}_{t} - {T}_{t} \vert}{{\sum }_{t = 1}^{n} {R}_{t}} \times 100$$

$${f}_{2} = 50 \times log \frac{100}{{[ 1 + [ ( {\sum }_{t = 1}^{n} {( {R}_{t} - {T}_{t} )}^{2} ) / n ] ]}^{- 0.5}}$$

Where, n is the number of release sample time intervals, Rₜ, and Tₜ are the percent released at each time point, t, for the reference and test drug release profiles, respectively.

Stability Study

Stability of the prepared liposomal formulations was examined at a temperature of 4±1° C. for 6 months, according to the guideline of the International Conference on Harmonisation (G. for Industry, −Q1A(R2) Stability Testing of New Drug Substances and Products, 2003). The stored samples were tested for their physical changes, particle size distribution, zeta potential, and EE %.

In-Vitro Cytotoxicity Study

The human mammary gland adenocarcinoma cell line, MCF-7; human liver hepatocellular carcinoma, HepG2; and human kidney normal cells, BHK-21 were exposed to variable concentrations of oxaliplatin. MTT assays were used to evaluate the cells viability as previously reported (Riss et al., Cell Viability Assays Assay Guidance Manual, Assay Guid. Man., pp. 1-23, 2004; Roehm et al., An improved colorimetric assay for cell proliferation and viability utilizing the tetrazolium salt XTT, J. Immunol. Methods, vol. 142, no. 2, pp. 257-265, 1991), which utilize the conversion of the tetrazolium salt MTT to formazan by dehydrogenase enzymes in living cells. Briefly, cells were cultured in 96-well plate (10,000 cells per well) at 37° C. humidified with 5% CO₂ in DMEM supplemented with 10% FBS and 5% Penicillin-Streptomycin mixture. Dilutions of oxaliplatin and liposomal formulations at 8, 12, 16, 20, and 28 μg/ml were made in the culture media. Samples were incubated with the cell line for 24 hrs, and wells containing cells treated only with media served as controls. After incubation, the media was discarded and the cells were further incubated in 20 μl MTT (5 mg/ml) and 100 μl fresh media for 3 hours, all media was then discarded, and the formazan crystalline precipitate formed were solubilized via the addition of 100 μl DMSO. The absorbance of each well was measured at 450 nm using a microplate reader, and the reference absorbance measured at 620 nm. Cell viability was determined by calculating the absorbance of the test wells as a percentage of the control wells. GraphPad prism 6 software package was used for calculation of IC₅₀.

Absorbance of well=A₆₂₀−A₄₅₀

Cell viability(%)=(Absorbance of experimental group/Absorbance of control untreated group)*100

### γ-H2AX Assay

MCF-7 cell line for human mammary gland adenocarcinoma was exposed to 2 μM concentration of free oxaliplatin, LP-Ox, LP-Ox-Stp and Lipoxal for 1 hr. After incubation, the media was discarded and the cells were further incubated in fresh media for 24 hours. Cells were then fixed using formaldehyde and permeabilized with Triton X-100, and subsequently incubated with H2AX primary antibody for 1 hr. Then, cells were washed using PBS and were further incubated for 30 min. in FITC mouse secondary antibody and washed using PBS. Finally cells were stained using DAPI and observed under a fluorescence microscope.

Statistical Analysis

All values are expressed as mean±S.D. Statistical analysis was performed using a two-tailed unpaired t-test, Tukey honest significant difference test, one-way ANOVA, two way ANOVA and linear and non-linear regression.

Results

Characterization of Liposomes

Original Formulation

With the aim of studying the effect of drug loading on the prepared liposomal system, three characteristic variables were studied for LP-void i.e. unloaded liposomes and oxaliplatin loaded liposomal formulation, LP-Ox. It was noted that the addition of oxaliplatin alter the PDI, and δ potential of liposomes significantly (P<0.001), while the particle size did not change significantly; refer to Table 5.

Phosphatidyl Glycerol Addition

As reported in Table 6, the replacement of DSPE with DSPG (LP-G1-Ox) had a significant influence on reducing the size of liposomes (P<0.001), and on enhancing the encapsulation efficiency of oxaliplatin. The addition of DSPG at the expense of cholesterol (LP-G3-Ox) was found to result in a significantly larger liposomal size (P<0.001), the same effect was observed but to a less extent upon displacing 5 mole % of DSPC with DSPG (LP-G2-Ox) (P<0.05). As for the effect of DSPG on potential, the incorporation of DSPG within the liposome had a significant reducing effect on ζ6 potential of all liposomal formulations (P<0.001). In addition, oxaliplatin loading was associated with a significant decrease in ζ potential in all DSPG containing liposomes (P<0.001).

Dual Drug Loading

Dual drug loaded liposomes with either ascorbic acid or satraplatin along with oxaliplatin had a direct influence on the final size, ζ potential, and EE % of oxaliplatin. The additional loading of ascorbic acid resulted in a significant increase in liposome size, and a subsequent increase in oxaliplatin EE %, while reduced the liposome's ζ potential. On the other hand, the additional loading of satraplatin was associated with reduction in liposome size, and an increase in liposomal ζ potential (see Table 7). LP-Ox-Stp had contradicting results in EE % calculated using ultracentrifugation (UC) and pellet permeabilization (PP) techniques. As UC-EE % determined a decrease in encapsulated oxaliplatin upon co-loading of satraplatin, while PP-EE % determined a direct relationship between oxaliplatin and satraplatin encapsulation. These results indicate that the dual loading of ascorbic acid results in enhancing the encapsulation of oxaliplatin within liposomes, while the dual loading of satraplatin influence on oxaliplatin encapsulation has contradictory results upon calculation of EE % using UC and PP protocols.

In addition, upon comparing the single drug loaded liposomes with satraplatin to dual drug loaded liposomes with satraplatin and oxaliplatin, a significant influence was observed in size reduction and increase in ζ potential for dual drug loaded liposomal system, with P<0.001, and P<0.01 respectively; and a minor influence of oxaliplatin on the EE % of satraplatin was observed.

Transmission Electron Microscopy (TEM)

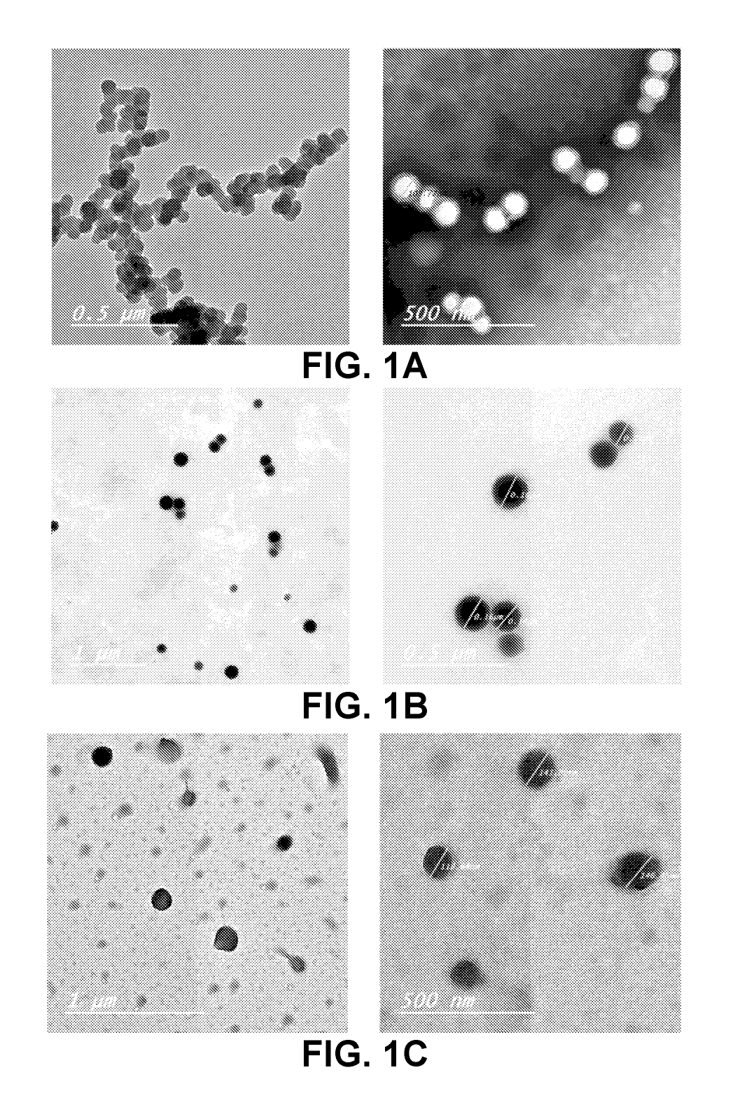

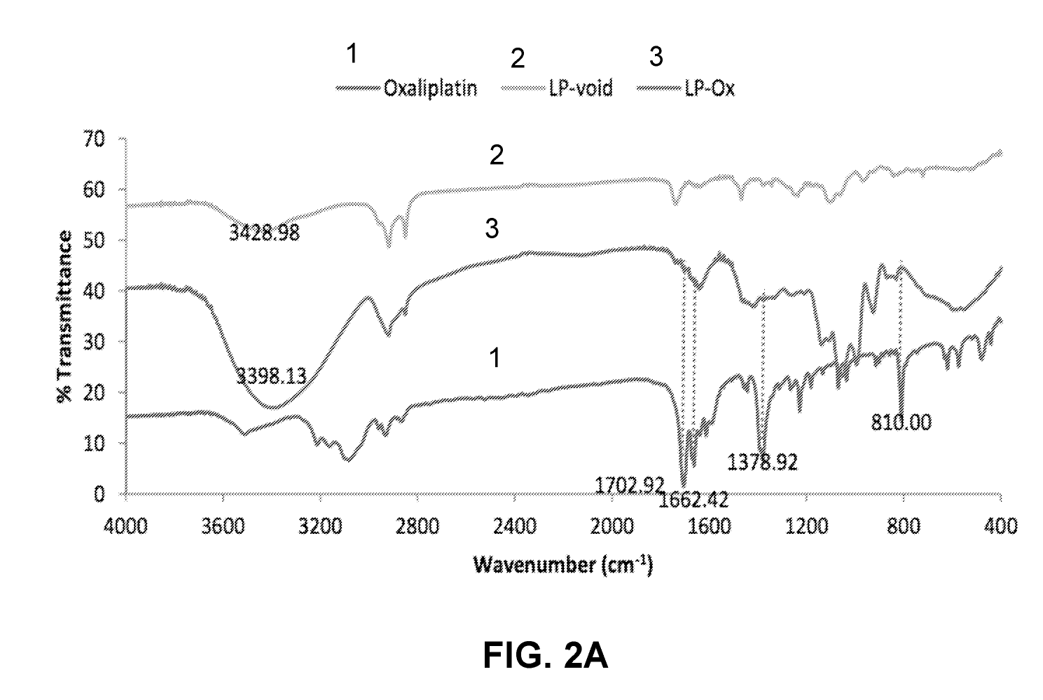

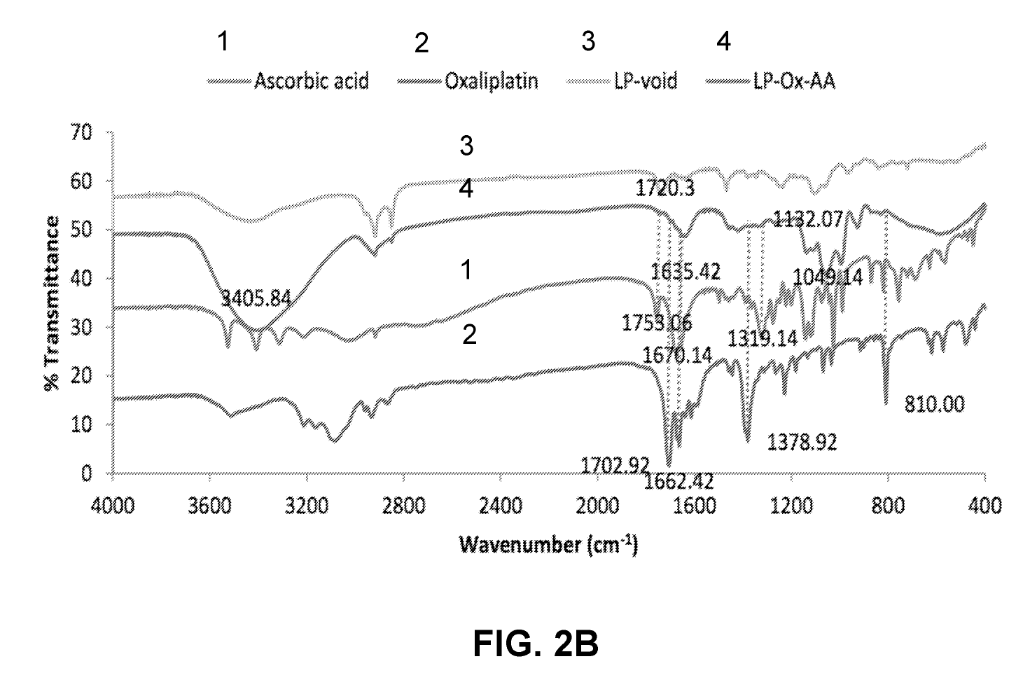

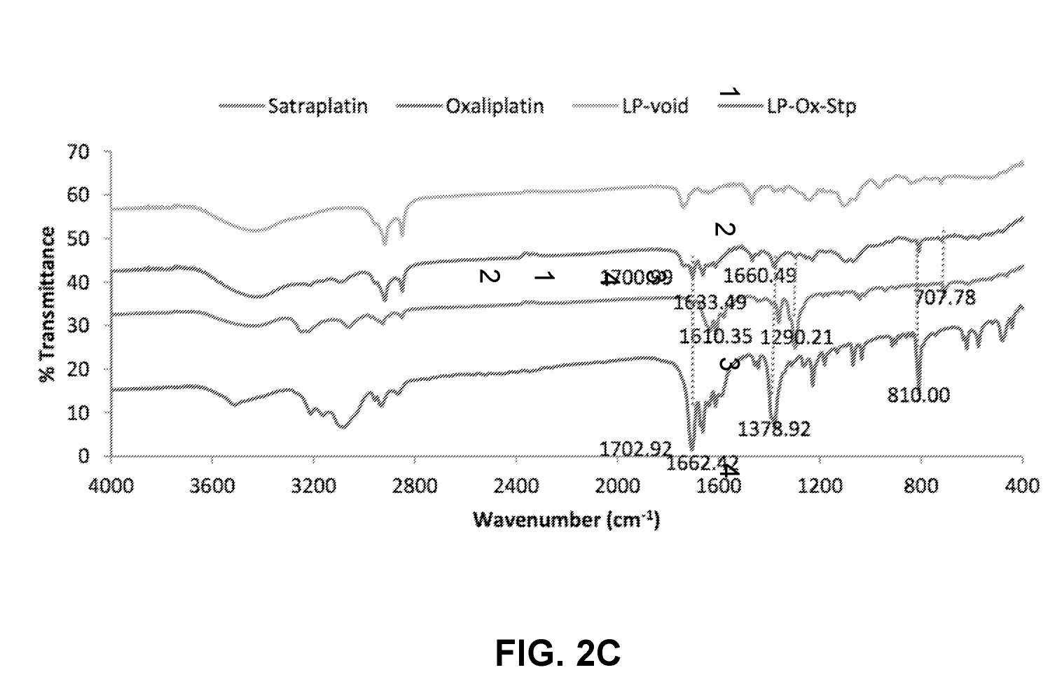

The morphology of liposomes was evaluated using TEM, which in turn has indicated the spherical structure for most liposomes with uniform particle size and uniform dispersion, as in FIGS. 1A-C. A white coated film was observed on the surface of the prepared liposomes that is attributed to the PEG coat over the surface of the liposomes, acting as a steric hindrant to mononuclear phagocytic cells of the RES.

The FT-IR spectrum comparison for the drug loaded liposomal formulations versus their unloaded liposomes and free drug spectra has revealed that no change in chemical structure occurred during the preparation of satraplatin oxaliplatin dual drug loaded liposomes LP-Ox-Stp, while disappearance of the carbonyl group was noted for the LP-Ox and LP-Ox-AA liposomal formulations indicates the involvement of carbonyl groups in hydrogen bonding, confirmed by the broad hydroxyl peak observed at 3300-3500 cm⁻¹.

Stability Study

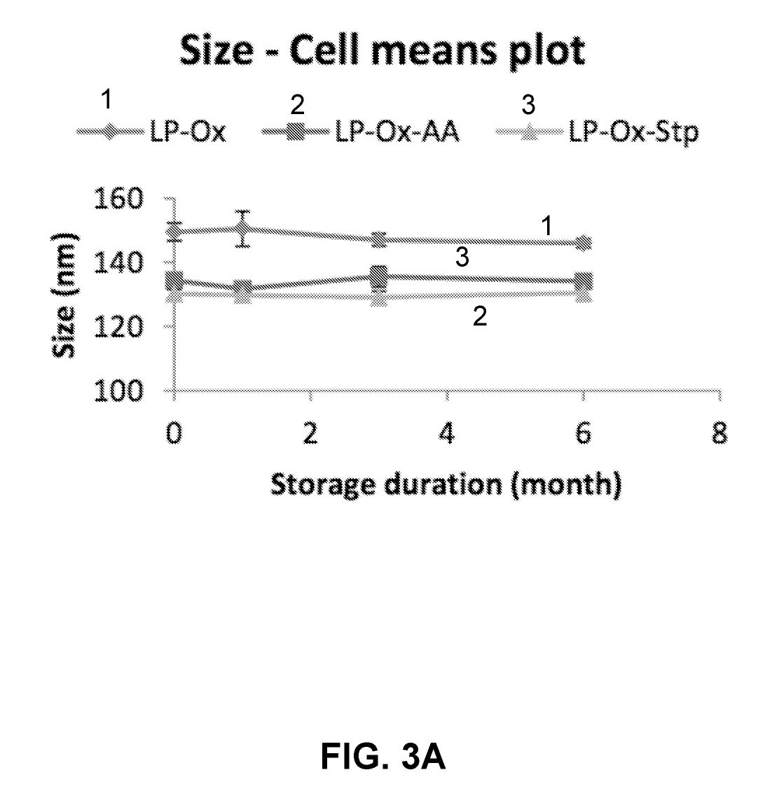

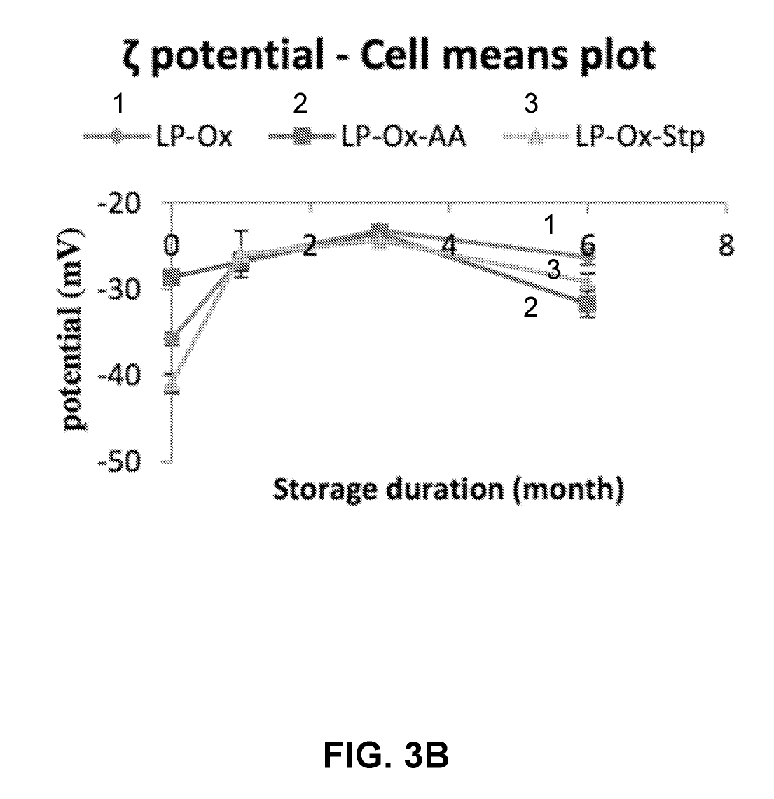

Relying on a two-way ANOVA analysis of the formulation stability data, it was found that the 6 month storage duration had no significant influence on the size of liposomes, but had a significant influence on ζ potential and PDI, P<0.001, P<0.01 respectively. In addition, there was a significant difference between all liposomal formulations in the variability of size, ζ potential and PDI during the 6 month storage duration, with P<0.001, P<0.001, and P<0.01 respectively. As illustrated in Table 8 and FIG. 3, the type of drug loaded within the liposomal formulation had a significant influence on its stability during a six month storage duration which was significantly interpreted in size and ζ potential P<0.001. LP-Ox loaded only with oxaliplatin had a gradual yet non-significant decrease in size along with a significant decrease in ζ potential upon storage (P<0.001) associated with an increase in the UC-EE %, and a decrease in the PP-EE %. Whereas dual drug loaded liposomes LP-Ox-AA and LP-Ox-Stp had a more stable size with minimal non-significant variations. LP-Ox-Stp showed a significant gradual decrease in ζ potential, with P<0.001 upon storage for 6 month and an increase in UC-EE % for oxaliplatin associated with a concomitant decrease in UC-EE % of satraplatin, while the PP-EE % has shown a significant decrease in both oxaliplatin and satraplatin encapsulation. LP-Ox-AA had no significant difference in ζ potential after 6 month storage at 4° C., and a significant decrease in UC-EE % and PP-EE % of oxaliplatin and ascorbic acid after 6 month storage. In addition it was noted that for the stability evaluation for LP-Ox-Stp after an 8 month storage duration indicates high stability of the formulation.

In-Vitro Drug Release Profile

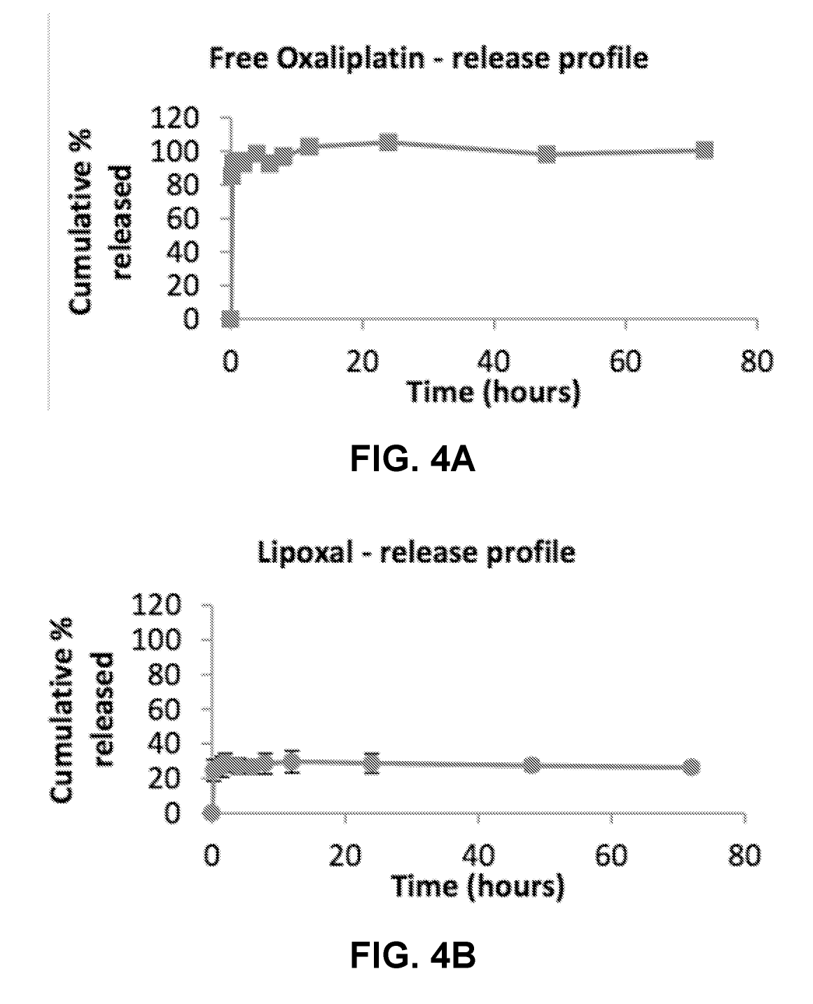

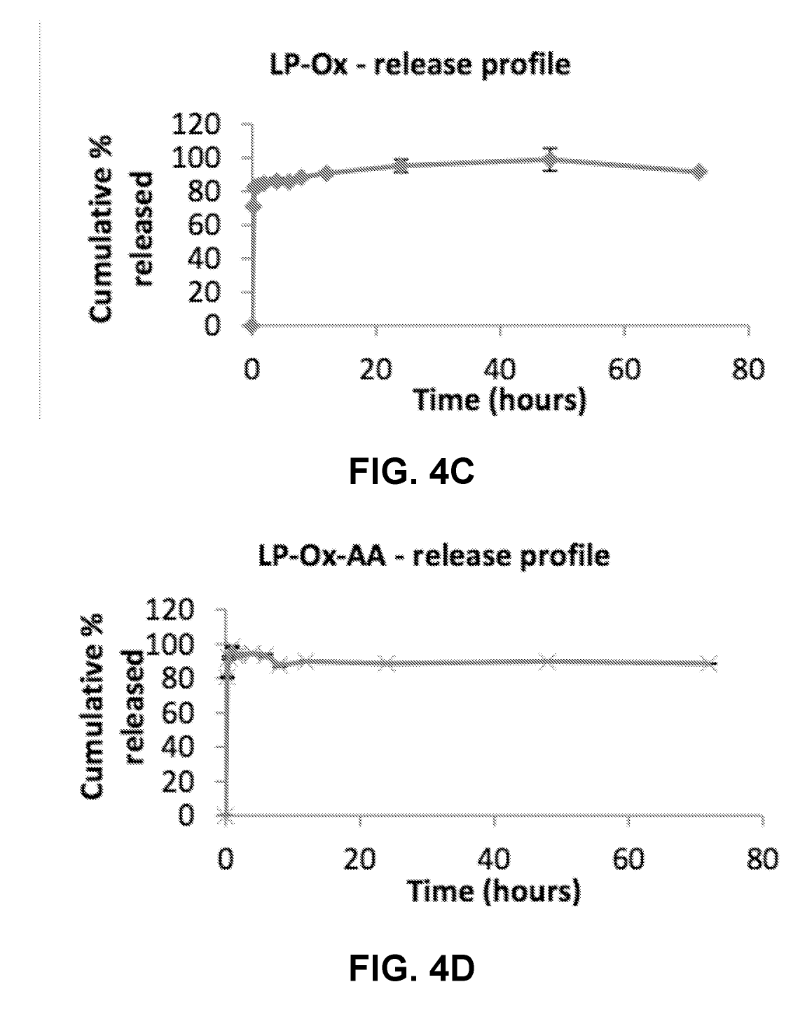

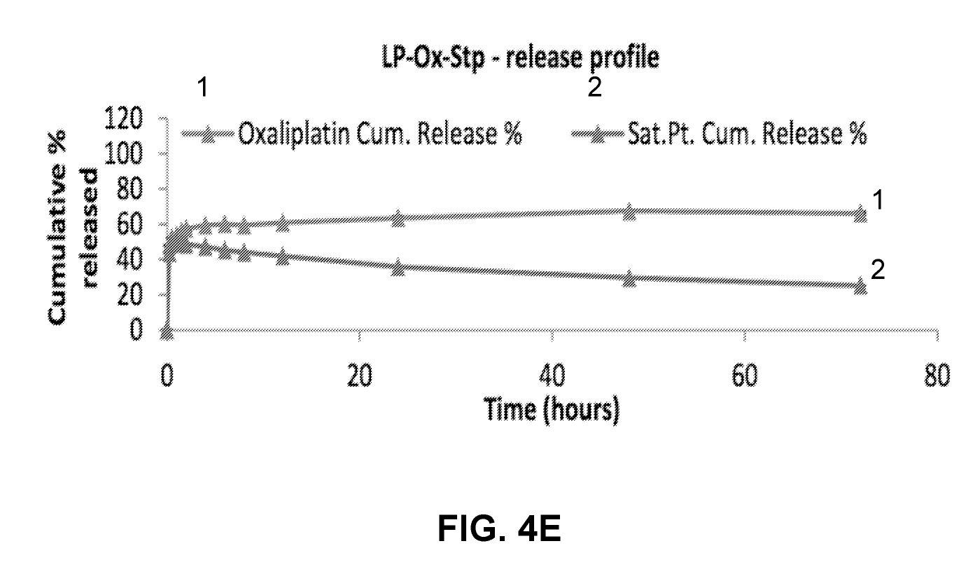

The drug release profiles for the prepared liposomal formulations are illustrated in FIGS. 4A-E. To further understand the differences in release profiles and their underlying cause, the drug release profiles for the three prepared liposomal formulation LP-Ox, LP-Ox-AA, LP-Ox-Stp were evaluated relative to free oxaliplatin drug solution, Oxaliplatin spiked liposomal formulation LP-void and LP-Stp, and a commercial oxaliplatin liposomal formulation, Lipoxal.

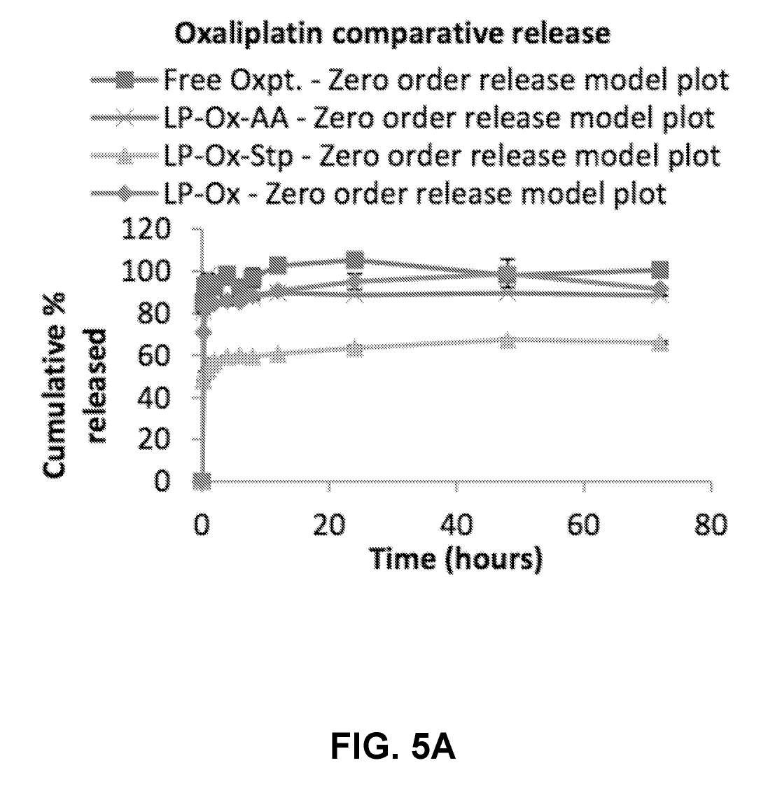

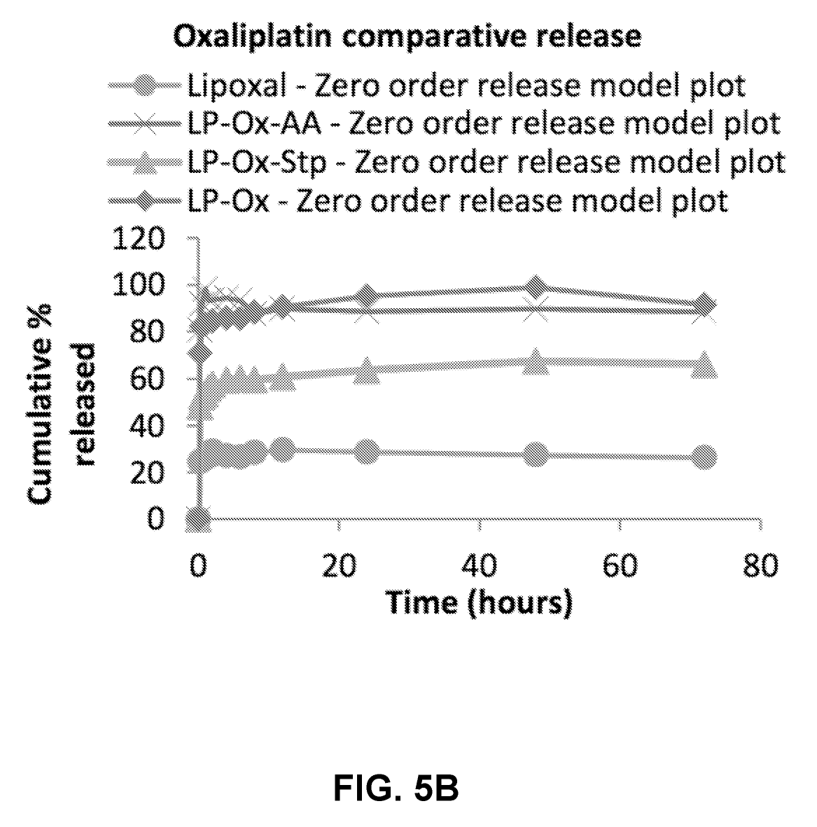

Relative to the release profile of free oxaliplatin, LP-Ox-Stp was the only formulation having an oxaliplatin release profile significantly different from free oxaliplatin (P<0.01) showing the least cumulative % release of oxaliplatin, i.e. a more efficient system for controlled release, as illustrated in FIG. 5A. Upon comparison with Lipoxal drug release profile, it was found that Lipoxal has a significantly different release profile from all of the liposomal formulations prepared (P<0.001); however, it was noted that LP-Ox-Stp had the least significant difference from oxaliplatin release profile to that of Lipoxal (P<0.05), refer to FIG. 5B. In addition it was noted that the co-loading of ascorbic acid with oxaliplatin had no significant influence on the rate of oxaliplatin release from the liposomal system.

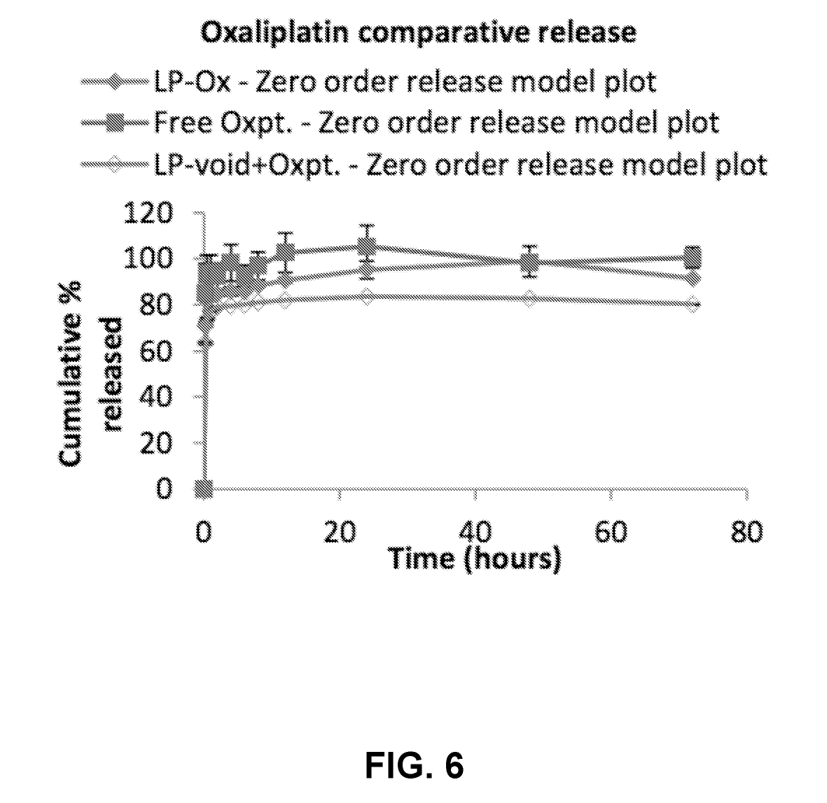

In an attempt to further understand the differences in the drug release profile obtained for the prepared liposomal formulations, an oxaliplatin spiked void liposomal formulation was used to examine its difference in terms of drug release from an oxaliplatin loaded liposomal formulation, LP-Ox, and free oxaliplatin solution (FIG. 6). It was noted that there is no significant difference in the release profile between all three of them. A possible explanation for the similarity in oxaliplatin release profile between drug loaded liposomes (LP-Ox), and free oxaliplatin would be the rapid release of entrapped drug; as for the similarity of free drug release profile with that of spiked liposomes (LP-void+Oxpt.) outweigh the absence of any drug binding to the liposomal bilayer.

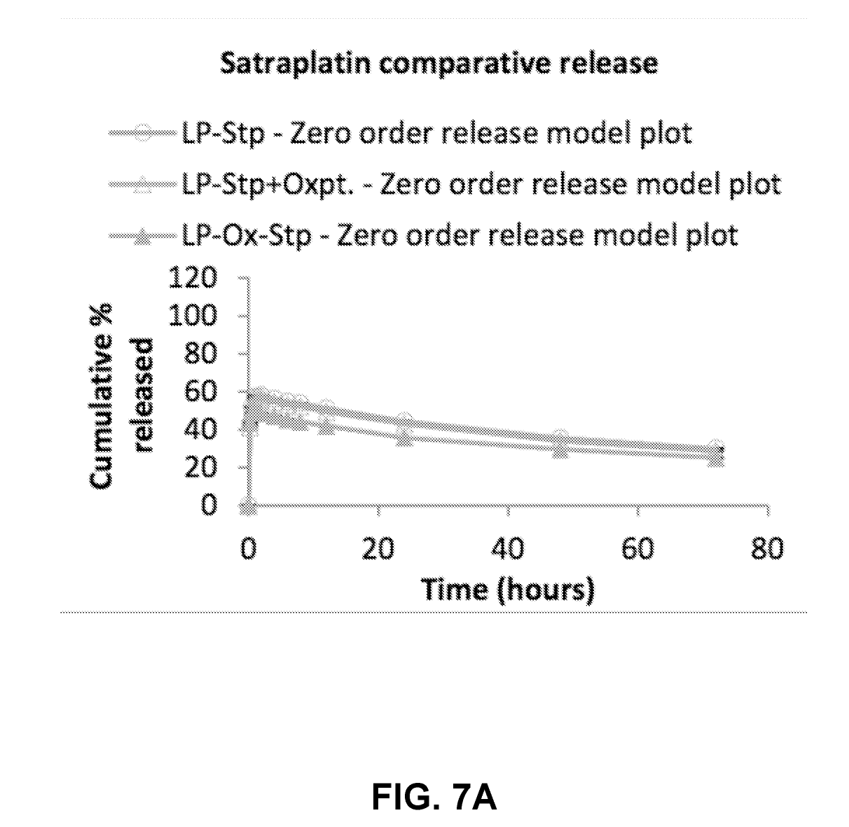

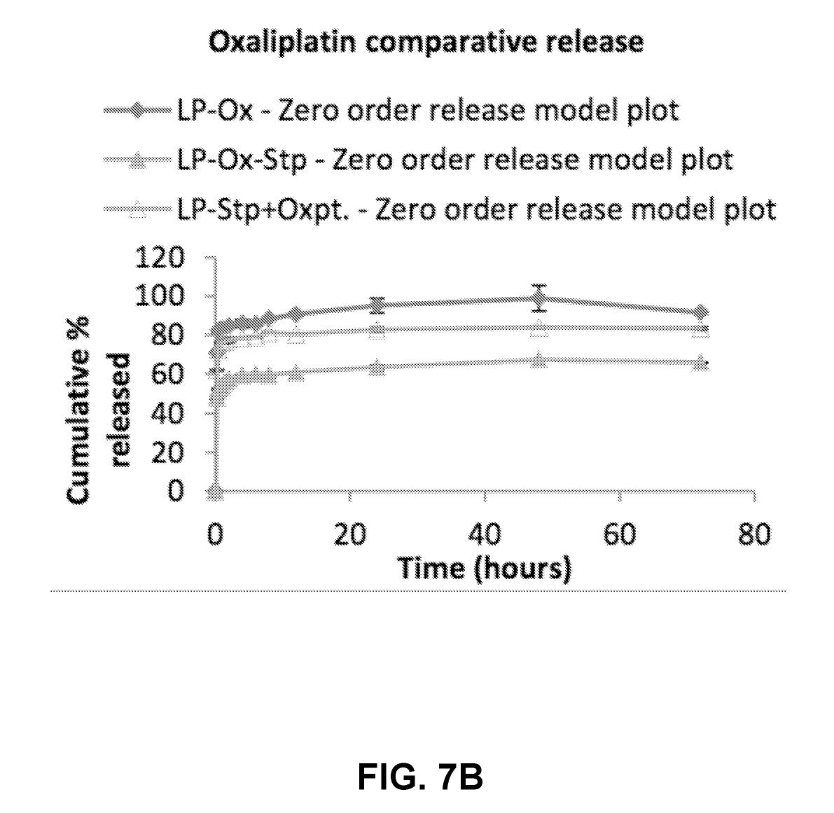

Similarly, the dual drug loaded liposomal formulation LP-Ox-Stp was examined for its satraplatin and oxaliplatin release profiles relative to satraplatin loaded liposomes (LP-Stp), satraplatin loaded liposomes spiked with oxaliplatin (LP-Stp+Oxpt.), and oxaliplatin loaded liposomes (LP-Ox). In the case of satraplatin release profile, no significant difference was noted between dual drug loaded liposome, single drug loaded liposome, and single drug loaded liposome spiked with oxaliplatin as illustrated in FIG. 7A. On the contrary, oxaliplatin release profile was significantly different for the dual drug loaded liposome, LP-Ox-Stp, compared to single drug loaded LP-Ox, and oxaliplatin spiked satraplatin loaded liposomes, LP-Stp+Oxpt (P<0.05), refer to FIG. 7B. Thus, it can be concluded that satraplatin co-loading with oxaliplatin in a liposomal system has a significant retarding influence on the release of oxaliplatin (P<0.05). That could be due to one of the following reasons, either as a result of reduced permeability of the liposome lipid bilayer to oxaliplatin, or due to enhanced binding of oxaliplatin to the lipid bilayer. However, the lack of significant difference between the oxaliplatin spiked LP-Stp liposomes and oxaliplatin loaded liposomes negates the second reason, and outweigh the reduced liposome permeability to oxaliplatin as a result of satraplatin accommodation in the lipid bilayer.

This comparative analysis of the release data was further validated by the calculation of the similarity and difference factors, f₂ and f₁, for the release data in the same comparison pattern used, reported in Table 9. Taking into consideration that samples are considered different if f₁>15, and f₂<50, free oxaliplatin was found to have a different oxaliplatin release profile from that of Lipoxal, LP-Ox-Stp, and LP-Stp+Oxpt; while maintaining a similar oxaliplatin release profile to LP-Ox, and LP-Ox-AA. Lipoxal had a different oxaliplatin release profile from free drug and all liposomal formulations. LP-Ox had a similar oxaliplatin release profile to that of free drug, and LP-void+Oxpt. LP-Ox-Stp had a different oxaliplatin release profile from LP-Ox, and LP-Stp+Oxpt; and a similar satraplatin release profile to both LP-Stp, and LP-Stp+Oxpt.

Release Kinetics

Drug release data were fitted to seven dissolution-diffusion kinetic models (zero-order, first-order, Higuchi, Hixon-Crowell, Korsmeyer-Peppas, Baker-Lonsdale, and Michaelis-Menten), and their respective kinetic parameters and coefficient of determination (R²) were calculated, refer to Table 10. In general, the zero-order, first-order, Higuchi, Hixson models were not suitable to explain the controlled drug release pattern obtained in this study. The plots had poor linear fit with low P-value and low coefficient of determination (R²<0.8). However, the Korsmeyer-Peppas, and Baker Lonsdale models had a perfect linear fit with the oxaliplatin release data for samples LP-Ox-Stp and LP-Stp+Oxpt, respectively (R²>0.9, P<0.001). However the value of n the release exponent was found to be beyond the limits of korsmeyer-peppas model.

On the other hand, the two parameter, rectangular hyperbola model was found to fit the release data for all formulations perfectly with R²>0.9, P<0.001; except for the satraplatin release data from LP-Ox-Stp and LP-Stp, and oxaliplatin release from Lipoxal, with R²<0.8, P<0.001. The hyperbolic release pattern indicates that the rate of drug release is not dependent on the concentration.

In-Vitro Cytotoxic Study

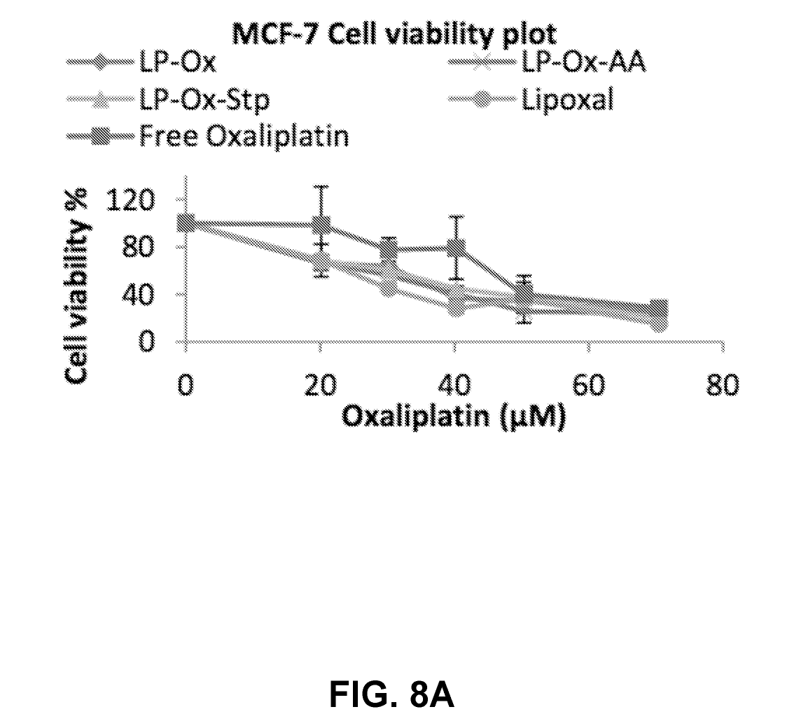

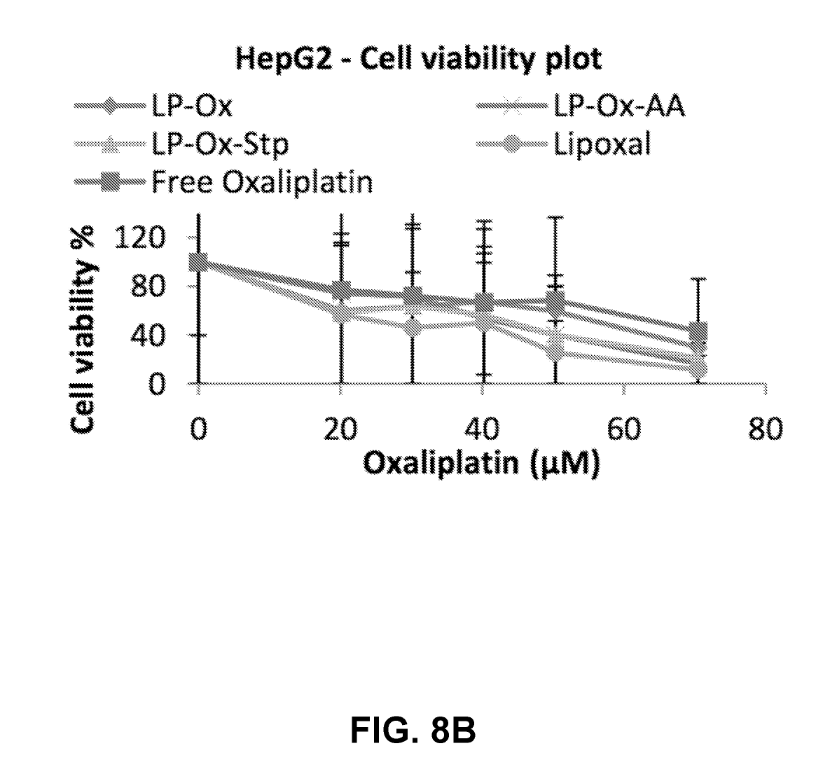

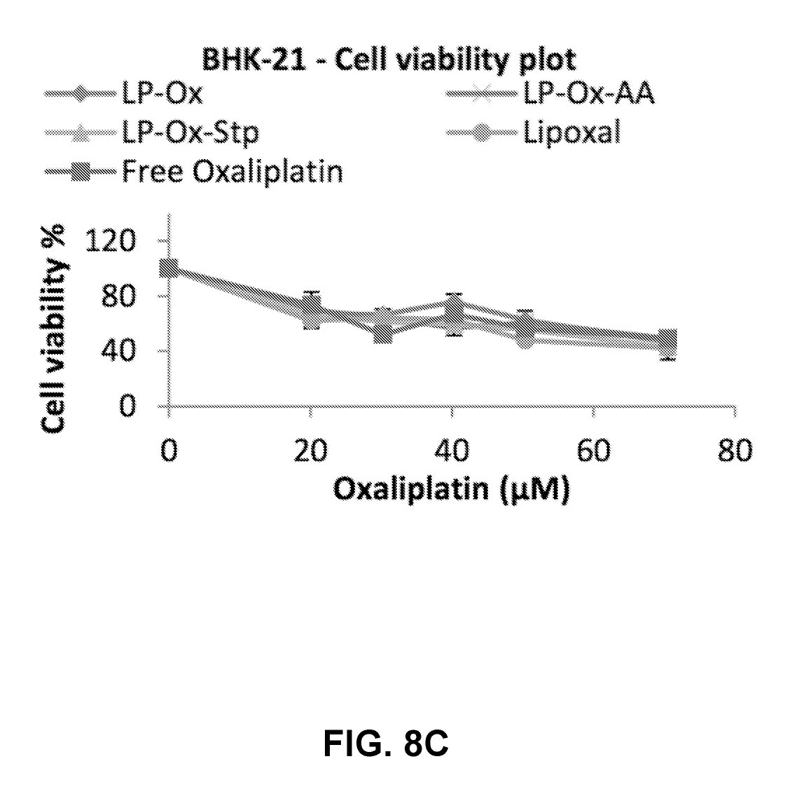

The cytotoxicity of the prepared liposomal formulations was examined on two cancer cell lines HepG2 and MCF-7 and one normal cell line BHK-21. In MCF-7, all tested liposomal formulations were found to cause a significantly higher cytotoxic effect than free oxaliplatin; with the following respective P-values LP-Ox, P<0.01; LP-Ox-AA, P<0.001; LP-Ox-Stp P<0.01; and Lipoxal, P<0.001 (FIG. 8A). Similarly in HepG2 cell line, the cytotoxic effect of all liposomal formulations was significantly higher than free oxaliplatin with P<0.001, except for LP-Ox P<0.05 (FIG. 8B). In addition, Lipoxal has shown a significantly higher cytotoxic effect in HepG2 cells over other liposomal formulations with P<0.001, except for LP-Ox-Stp P<0.01 (FIG. 8B). On the contrary to cancer cells, there was no significant difference in cytotoxic effect between all tested formulations on normal cells, BHK-21; all formulations had an overall much weaker cytotoxic effect over normal cells relative to cancer cells (FIG. 8C).

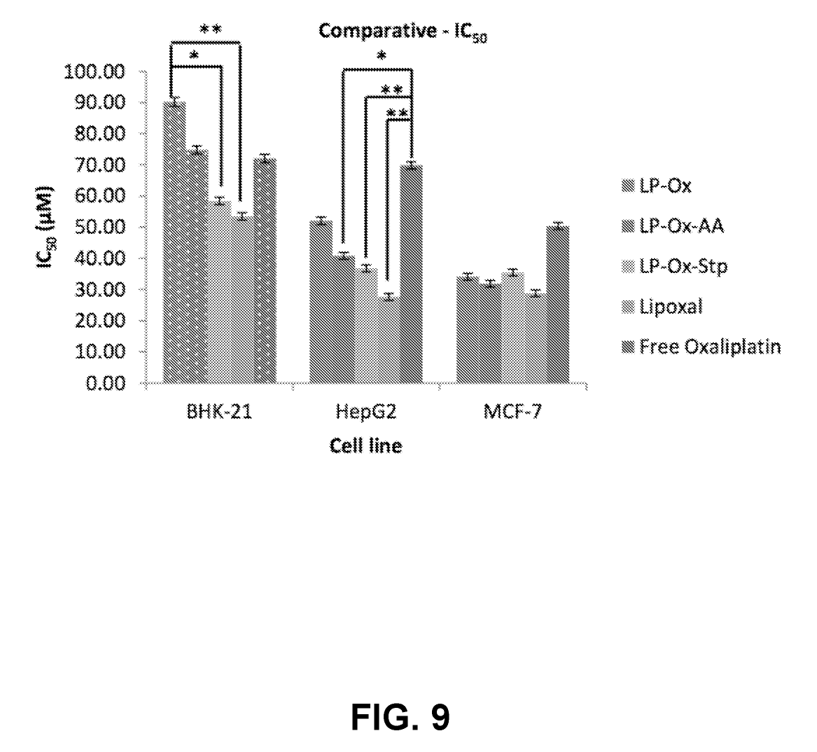

Upon comparing the IC₅₀ of the tested formulations in each cell line, it was found that the Lipoxal, LP-Ox-AA, and LP-Ox-Stp had a significantly lower IC₅₀ relative to free oxaliplatin, P<0.01, in HepG2 cell line; and in BHK-21 cell line LP-Ox-Stp and Lipoxal had a significantly lower IC₅₀ relative to LP-Ox with P<0.05, and P<0.01 respectively (FIG. 9). However the IC₅₀ values obtained for LP-Ox, Free drug, and LP-Ox-AA in BHK-21 are extrapolated from cell viability data at lower concentrations since they are out of the oxaliplatin concentration range used in the experiment (0-28 μg/ml) (Table 11).

DNA Damage Preliminary Data

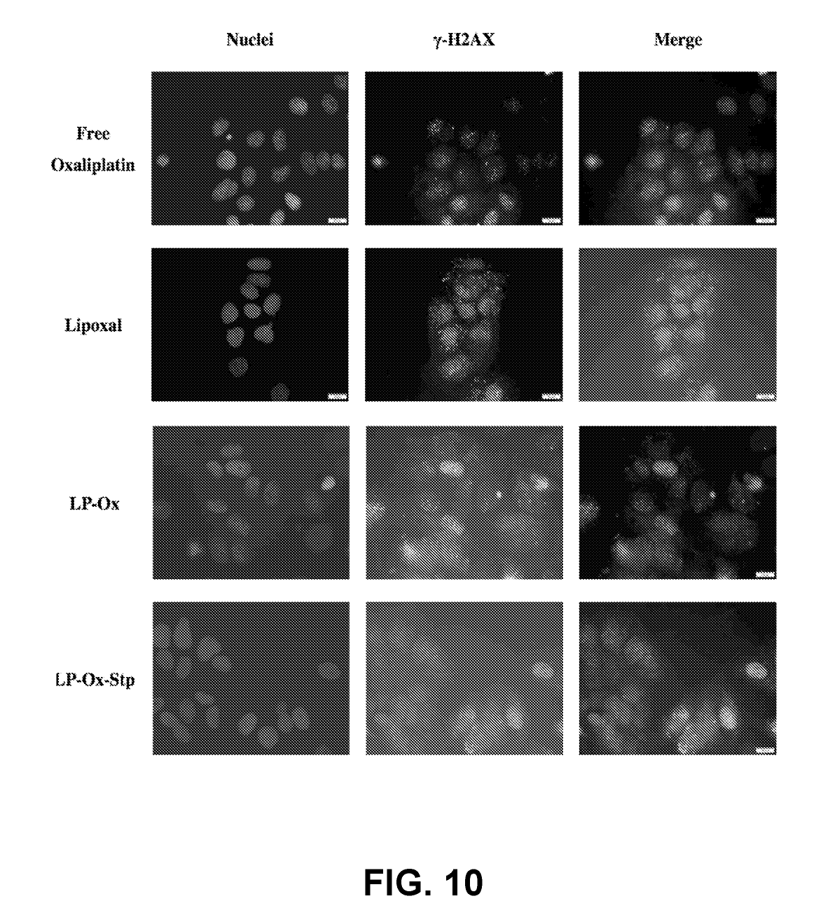

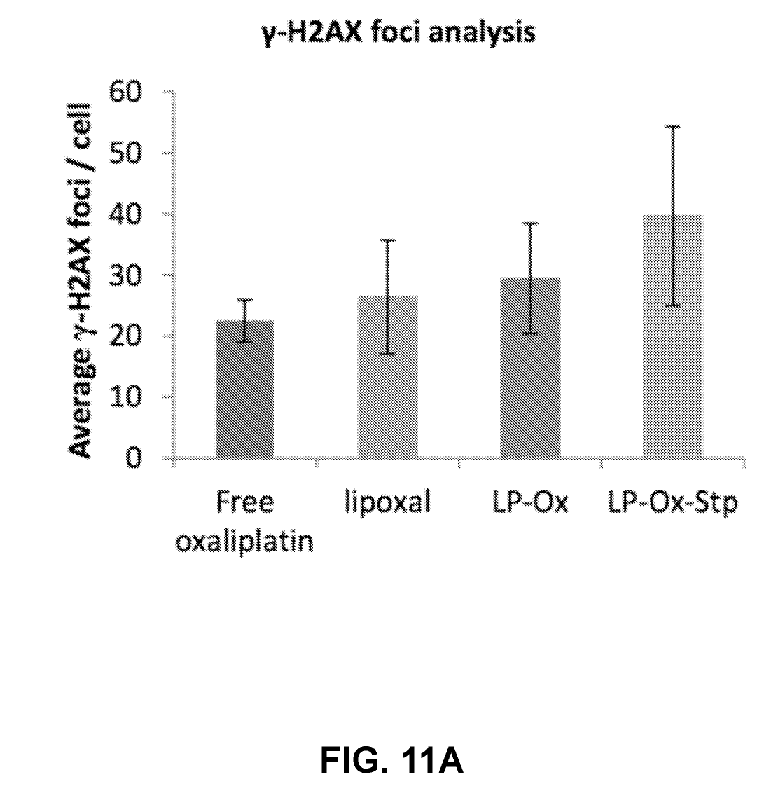

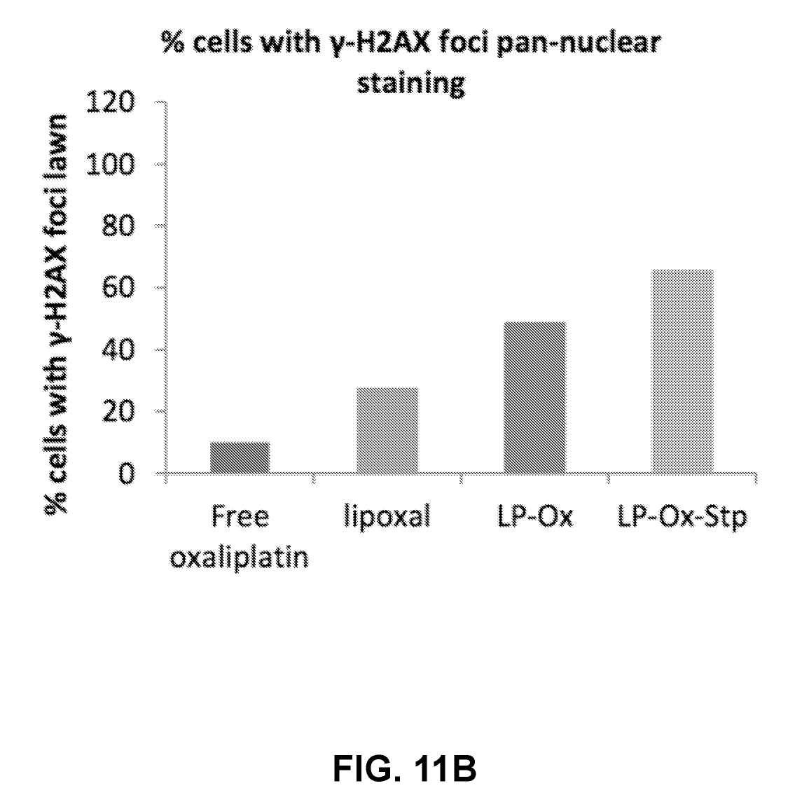

Free oxaliplatin resulted in a relatively lower DNA damage as indicated from its immunofluorescence images showing few γ-H2AX foci and minimal pan-nuclear staining similar to Lipoxal and LP-Ox but with a slightly higher magnitude of DNA damage (FIG. 10, 4 11A-B). Whereas LP-Ox-Stp demonstrated the highest DNA damage magnitude, exceeding 60% foci pan-nuclear staining.

TABLE 1

| | LIPID | | | | Transition |
|---|---|---|---|---|---|
| Lipid | MAPS | IUPAC | Structure | Charge | Temperature |
| DSPC | 18:0 PC | (2R)-2,3-Bis(stearoyloxy)propyl 2- (trimethylammonio)ethyl phosphate | 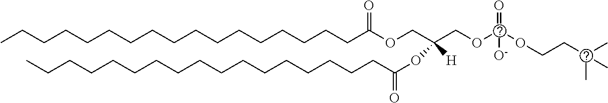 | Zwitterion | 55 |
| DSPE | 18:0 PE | 3-{[(2- Aminoethoxy)(hydroxy)phosphoryl]oxy}- 2-(stearoyloxy)propyl stearate | 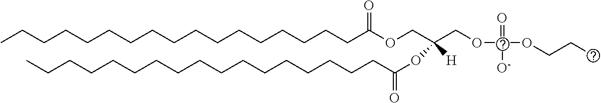 | Zwitterion | 74 |
| DSPG | 18:0 PG | 3-{[(2,3- Dihydroxypropoxy)(hydroxy)phosphoryl]oxy}- 2-(stearoyloxy)propyl stearate | 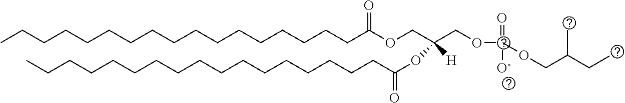 | Anionic | 55 |
| Cholesterol | Cholesterol | (3β)-Cholest-5-en-3-ol | 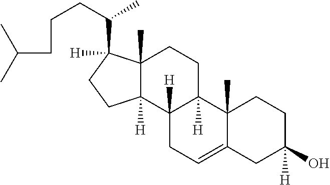 | Neutral | NA |
| MPEG- 2000-DSPE | 18:0 PEG2000 PE | 1,2-distearolyl-sn-glycero-3- phosphoethanolamine-N- [methoxy(polyethylene glycol)-2000] (sodium salt) | 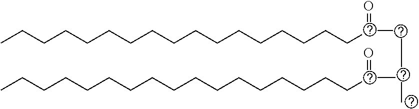 [SMILES: CCCCCCCCCCCCCCCCCC(=O)CC(C)C(=O)CCCCCCCCCCCCCCCCC] | Anionic | NA |

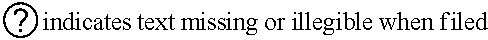

TABLE 2

| | | | | | | Total lipid | Lipid | Oxaliplatin | |
|---|---|---|---|---|---|---|---|---|---|
| | | | | | | number of | Concentration | | number of |
| | | | | | DSPE- | moles | (LC) | Mole ratio | moles |
| Sample | DSPC | Cholesterol | DSPE | DSPG | PEG2000 | (mmole) | (mM) | Drug:Lipid | (mmole) |
| LP | 1.000 | 0.8000 | 0.1000 | — | 1.100 | 0.0412 | 13.75 | 167:304 | 0.022653 |
| LP-Void | 1.000 | 0.8000 | 0.1000 | — | 1.100 | 0.0412 | 13.75 | 0:1 | 0 |
| LP-G1 | 1.000 | 0.8000 | — | 0.1000 | 0.1000 | 0.0412 | 13.75 | 167:304 | 0.022653 |
| LP-G2 | 1.000 | 0.8889 | 0.1111 | 0.1111 | 0.1111 | 0.0412 | 13.75 | 167:304 | 0.022653 |
| LP-G3 | 1.000 | 0.7000 | 0.1000 | 0.1000 | 0.1000 | 0.0412 | 13.75 | 167:304 | 0.022653 |

TABLE 3

| | | | | | Total lipid | | Oxaliplatin | | Hydrophilic/Hydrophobic drug | | |
|---|---|---|---|---|---|---|---|---|---|---|---|
| | | | | | number of | | | number of | | Percent | number of |
| | | | | DSPE- | moles | LC | Mole ratio | moles | Mole ratio | of total | moles |
| Sample | DSPC | Cholesterol | DSPE | PEG2000 | (mmole) | (mM) | Drug:Lipid | (mmole) | Drug:Lipid | lipid | (mmole) |
| LP-Ox-AA | 1.000 | 0.8000 | 0.1000 | 1.100 | 0.0412 | 13.75 | 167:304 | 0.022653 | 167:304 | — | 0.0227 |
| LP-Ox-Stp | 1.000 | 0.8000 | 0.1000 | 1.100 | 0.0412 | 13.75 | 167:304 | 0.022653 | — | 10 | 0.0046 |
| LP-Stp | 1.000 | 0.8000 | 0.1000 | 1.100 | 0.0412 | 13.75 | — | — | — | 10 | 0.0046 |

TABLE 4

| | | | Cell membrane |
|---|---|---|---|
| | | Log P | permeability |
| Drug name | IUPAC name | (Chemaxon) | (cm/s) |
| Oxaliplatin | Platinum(2+) ethanedioate (1R,2R)-1,2-cyclohexanediamine (1:1:1) | 1.73 | <1 × 10⁻⁶ [91] |
| Ascorbic acid | (5R)-5-[(1S)-1,2-Dihydroxyethyl]-3,4-dihydroxy-2(5H)-furanone | −1.91 | — |
| Satraplatin | Bis(acetato-κO)(ammine)dichloro(cyclohexanamine)platinum | 1.17 | — |

TABLE 5

| | Size | | | | ζ Potential | |
|---|---|---|---|---|---|---|
| Sample | (nm) | ±SD | PDI | ±SD | (mV) | ±SD |
| LP-void | 150.3 | 1.367 | 0.084 | ±0.020 | −40.72 | ±0.638 |
| LP-Ox | 149.5^{ns} | ±2.811 | 0.040*** | ±0.025 | −35.8*** | ±0.733 |

Data are means ± SD from n = 3.
***P < 0.001: difference from drug unloaded liposomal formulation, LP-void.
^{ns}not significant versus LP-void.

TABLE 6

| | | | | | | | Oxaliplatin | | Oxaliplatin | |
|---|---|---|---|---|---|---|---|---|---|---|
| | Size | | | | ζ Potential | | (Oxpt) | | (Oxpt) | |
| Sample | (nm) | ±SD | PDI | ±SD | (mV) | ±SD | UC-EE % | ±SD | PP-EE % | ±SD |
| LP-Ox | 149.5 | ±2.811 | 0.040 | ±0.025 | −35.8 | ±0.733 | 23.7 | ±3.860 | 8.217 | ±0.01738 |
| LP-G1-Ox | 136.7 [symbol] | ±1.183 | 0.056 | ±0.034 | −29.3 [symbol] | ±2.18 | 26.04 [symbol] | ±0.3571 | 11.90 [symbol] | ±0.1726 |
| LP-G2-Ox | 144.0 [symbol] | ±5.745 | 0.069 | ±0.039 | −29.4 [symbol] | ±2.05 | 24.11 | ±0.09466 | 9.007 [symbol] | ±0.1577 |
| LP-G3-Ox | 155.6 [symbol] | ±7.098 | 0.055 | ±0.026 | −28.5 [symbol] | ±1.43 | 25.11 | ±0.1194 | 14.55 [symbol] | ±0.1591 |
| LP-G1-void | 146.2 [symbol] | ±3.780 | 0.077 | ±0.026 | −57.6 [symbol] | ±1.47 | — | — | — | — |
| LP-G2-void | 149.6 [symbol] | ±4.555 | 0.123 | ±0.0541 | −50.2 [symbol] | ±2.51 | — | — | — | — |
| LP-G3-void | 146.5 [symbol] | ±5.927 | 0.069 | ±0.029 | −53.8 [symbol] | ±1.63 | — | — | — | — |

Data are means ± SD from n = 3. [symbol]
: P < 0.05 difference from liposomal formulation lacking DSPG, LP-Ox. [symbol]
: P < 0.001 difference from liposomal formulation lacking DSPG, LP-Ox. [symbol]
: P < 0.05 difference from liposomal formulation lacking DSPE, LP-G1-Ox. [symbol]
: P < 0.001 difference from liposomal formulation lacking DSPE, LP-G1-Ox. [symbol]
: P < 0.001 difference from liposomal formulation with DSPG added at the expense of DSPC, LP-G2-Ox. [symbol]
: P < 0.05 difference from liposomal formulation with DSPG added at the expense of DSPC, LP-G2-Ox. [symbol]
: P < 0.001 difference from liposomal formulation with DSPG added at the expense of Cholesterol, LP-G3-Ox. [symbol]
: P < 0.01 difference from liposomal formulation with DSPG added at the expense of Cholesterol, LP-G3-Ox. [symbol]
indicates data missing or illegible when filed

TABLE 7

| | | | | | ζ | | Oxpt | | AA/Stp | | Oxpt | | AA/Stp | |
|---|---|---|---|---|---|---|---|---|---|---|---|---|---|---|
| | Size | | | | Potential | | UC-EE | | UC-EE | | PP-EE | | PP-EE | |
| Sample | (nm) | ±SD | PDI | ±SD | (mV) | ±SD | % | ±SD | % | ±SD | % | ±SD | % | ±SD |
| Lipoxal | 118.5 | ±1.306 | 0.184 | ±0.0178 | −14.4 | ±1.66 | 53.99 | ±0.9310 | — | — | 7.390 | ±0.1255 | — | — |
| LP-Ox | 149.5 | ±2.811 | 0.040 | ±0.025 | −35.8 | ±0.733 | 23.70 | ±3.860 | — | — | 8.217 | ±0.01738 | — | — |
| LP-Ox- | 154.4 [symbol] | ±3.017 | 0.047 | ±0.028 | −22.3 [symbol] | ±0.817 | 28.54 [symbol] | ±3.593 | 97.81 | ±2.142 | 9.108 [symbol] | ±0.3825 | 46.19 | ±1.009 |
| AA | | | | | | | | | | | | | | |
| Lp-Ox- | 130.2 [symbol] | ±1.421 | 0.057 | ±0.026 | −40.9 [symbol] | ±1.14 | 16.75 [symbol] | ±4.177 | 49.34 [symbol] | ±2.550 | 19.10 [symbol] | ±0.05844 | 20.42 [symbol] | ±0.1160 |
| Stp | | | | | | | | | | | | | | |
| LP-Stp | 155.4 | ±1.425 | 0.070 | ±0.029 | −36.1 | ±1.94 | — | — | 48.32 | ±2.601 | — | — | 19.12 | ±0.07228 |

Data are means ± SD from n = 3. [symbol]
: P < 0.001 difference from single drug loaded liposomal formulation, LP-Ox. [symbol]
: P < 0.001 difference from dual drug loaded liposomal formulation, LP-Ox-AA. [symbol]
: P < 0.001 difference from single drug loaded liposomal formulation, LP-Stp. [symbol]
: P < 0.01 difference from single drug loaded liposomal formulation, LP-Stp. [symbol]
: P < 0.05 difference from single drug loaded liposomal formulation, LP-Stp. [symbol]
indicates data missing or illegible when filed

TABLE 8

| | | | | | | | ζ | | | | | | | | | |
|---|---|---|---|---|---|---|---|---|---|---|---|---|---|---|---|---|
| | Storage | | | | | | Poten- | | Oxpt | | AA/Stp | | Oxpt | | AA/Stp | |
| | duration | Physical | Size | | | | tial | | UC-EE | | UC-EE | | PP-EE | | PP-EE | |
| Sample | (month) | stability | (nm) | ±SD | PDI | ±SD | (mV) | ±SD | % | ±SD | % | ±SD | % | ±SD | % | ±SD |
| LP-Ox | 0 | No | 149.5 | ±2.81 | 0.040 | ±0.025 | −35.8 | ±0.733 | 23.70 | ±3.86 | — | — | 8.22 | ±0.017 | — | — |
| | 1 | Aggre- | 150.4 | ±5.45 | 0.032 | ±0.029 | −26.4 [symbol] | ±0.576 | | | | | | | | |
| | 3 | gates | 147.0 | ±1.95 | 0.046 | ±0.023 | −23.2 [symbol] | ±0.654 | | | | | | | | |
| | 6 | | 145.9 | ±1.38 | 0.048 | ±0.030 | −26.3 [symbol] | ±0.931 | 42.93 [symbol] | ±1.06 | — | — | 7.55 [symbol] | ±0.044 | — | — |
| | 8 [symbol] | | — | — | — | — | — | — | — | — | — | — | — | — | — | — |
| LP-Ox- | 0 | No | 134.3 | ±3.02 | 0.047 | ±0.028 | −22.3 | ±0.817 | 28.54 | ±3.59 | 97.81 | ±2.1 | 9.11 | ±0.383 | 46.19 | ±1.01 |
| AA | 1 | Aggre- | 131.6 | ±0.99 | 0.047 | ±0.022 | −26.9 | ±1.06 | | | | | | | | |
| | 3 | gates | 135.5 | ±3.18 | 0.072 | ±0.025 | −23.5 [symbol] | ±0.524 | | | | | | | | |
| | 6 | | 134.0 | ±2.03 | 0.081 | ±0.027 | −31.7 [symbol] | ±1.48 | 26.83 [symbol] | ±3.66 | 79.86 [symbol] | ±1.04 | 5.56 [symbol] | ±0.066 | 2.82 [symbol] | ±0.241 |
| | 8 | | 132.1 | ±0.99 | 0.10 | ±0.048 | −29.7 | ±0.694 | 28.28 | ±3.59 | 94.67 | ±0.43 | 9.52 | ±0.284 | 15.96 | ±1.203 |
| LP-Ox- | 0 | No | 130.2 | ±1.42 | 0.057 | ±0.026 | −40.9 | ±1.14 | 16.75 | ±4.18 | 49.34 | ±2.55 | 19.1 | ±0.058 | 20.42 | ±0.116 |
| StP | 1 | Aggre- | 129.7 | ±1.68 | 0.057 | ±0.023 | −25.9 [symbol] | ±2.70 | | | | | | | | |
| | 3 | gates | 129.1 | ±1.90 | 0.064 | ±0.015 | −24.4 [symbol] | ±0.241 | | | | | | | | |
| | 6 | | 130.4 | ±2.35 | 0.070 | ±0.038 | −29.1 [symbol] | ±0.904 | 32.21 [symbol] | ±3.40 | 46.32 [symbol] | ±2.78 | 11.8 [symbol] | ±0.072 | 11.36 [symbol] | ±0.051 |
| | 8 | | 128.4 | ±1.97 | 0.066 | ±0.028 | −27.6 | ±0.568 | 37.95 | ±3.18 | 54.59 | ±2.33 | 7.19 | ±0.161 | 8.208 | ±0.004 |

Data are means ± SD from n = 3. [symbol]
: P < 0.001 difference from 0 storage time. [symbol]
: P < 0.01 difference from 0 storage time. [symbol]
: P < 0.05 difference from 0 storage time. [symbol]
: P < 0.01 difference from 1 month storage duration. [symbol]
: P < 0.05 difference from 1 month storage duration. [symbol]
: P < 0.01 difference from 3 month storage duration. [symbol]
: P < 0.05 difference from 3 month storage duration. [symbol]
: The 8 month measurement was not evaluted for LP-Ox due to insufficient sample amount for 2 measurements at 8 and 12 month, thus the 12 month measurement was given a higher priority. [symbol]
indicates data missing or illegible when filed

TABLE 9

| | | Reference | | Reference | | Reference | | Reference | | Reference | | Reference | |
|---|---|---|---|---|---|---|---|---|---|---|---|---|---|
| | | Free Oxpt | | LP-void-Oxpt | | Lipoxal | | LP-Ox | | LP-Stp | | LP-Stp + Oxpt | |
| Formulations | | F1 | F2 | F1 | F2 | F1 | F2 | F1 | F2 | F1 | F2 | F1 | F2 |
| Lipoxal | Oxaliplatin | 71.65 | 8.940 | 65.27 | 15.34 | — | — | 68.39 | 12.11 | — | — | — | — |
| Free Drug | Oxaliplatin | — | — | 22.47 | 38.25 | 252.7 | 8.940 | 11.37 | 50.60 | — | — | — | — |
| LP-void + Oxpt | Oxaliplatin | 18.35 | 38.25 | — | — | 188.0 | 15.34 | 9.470 | 53.951 | — | — | — | — |
| LP-Ox | Oxaliplatin | 101.2 | 51.86 | 1.202 | 31.31 | 218.9 | 12.90 | — | — | — | — | — | — |
| LP-Ox-AA | Oxaliplatin | 5.482 | 55.25 | 15.76 | 44.62 | 233.3 | 10.66 | 4.124 | 54.73 | — | — | — | — |
| LP-Stp | Satraplatin | — | — | — | — | — | — | — | — | — | — | — | — |
| LP-Stp + Oxpt | Oxaliplatin | 18.99 | 37.47 | 0.787 | 91.76 | 185.7 | 15.57 | 10.48 | 51.95 | — | — | — | — |
| | Satraplatin | — | — | — | — | — | — | — | — | 2.884 | 91.13 | — | — |
| LP-Ox-Stp | Oxaliplatin | 38.94 | 22.13 | 25.21 | 35.81 | 115.35 | 25.76 | 32.54 | 28.19 | — | — | 24.62 | 36.59 |
| | Satraplatin | — | — | — | — | — | — | — | — | 14.25 | 56.50 | 11.70 | 60.60 |

TABLE 10

| | | Zero-order | | First-order | | Higuchi | | Hixson-Crowell | |
|---|---|---|---|---|---|---|---|---|---|
| Formulations | | [symbol] | K₀ | [symbol] | K₁ | [symbol] | K_{H} | [symbol] | K_{C} |
| Lipoxal | Oxaliplatin | 0.0378 | 0.0677 | 0.0377 | −0.0003 | 0.1302 | 1.0755 | 0.0365 | −0.0072 |
| Free Drug | Oxaliplatin | 0.0787 | 0.3444 | 0.4773 | −0.0172 | 0.1889 | 4.5644 | 0.0483 | −0.0127 |
| LP-void + | Oxaliplatin | 0.0739 | 0.2749 | 0.1229 | −0.0033 | 0.1891 | 3.7622 | 0.0476 | −0.0118 |
| Oxpt | | | | | | | | | |
| LP-Ox | Oxaliplatin | 0.1358 | 0.4224 | 0.3988 | −0.0127 | 0.2813 | 5.2102 | 0.0692 | −0.0148 |
| LP-Ox-AA | Oxaliplatin | 0.0213 | 0.1688 | 0.0027 | 0.0009 | 0.0801 | 2.7965 | 0.0305 | −0.0099 |
| LP-Stp | Satraplatin | 0.0987 | −0.2321 | 0.1638 | 0.002 | 0.0231 | −0.9608 | 0.0004 | 0.0009 |
| LP-Stp + | Oxaliplatin | 0.1064 | 0.3297 | 0.2228 | −0.0045 | 0.2344 | 4.1863 | 0.0562 | −0.0128 |
| Oxpt | Satraplatin | 0.1052 | −0.2347 | 0.1688 | 0.002 | 0.0273 | −1.0229 | 0.0007 | 0.0012 |
| LP-Ox-Stp | Oxaliplatin | 0.1766 | 0.326 | 0.2933 | −0.003 | 0.3279 | 3.8003 | 0.0728 | −0.0132 |
| | Satraplatin | 0.1308 | −0.2302 | 0.1949 | 0.0017 | 0.0536 | −1.2598 | 0.0017 | 0.0019 |

| | | Korsmeyer-peppas | | Baker-Lonsdale | | Michaelis-Menten | |
|---|---|---|---|---|---|---|---|
| Formulations | | [symbol] | n | [symbol] | K [symbol] | [symbol] | K [symbol] |
| Lipoxal | Oxaliplatin | 0.2858 | 0.0173 | 0.4898 | 0.0582 | 0.7873 | 0.0396 |
| Free Drug | Oxaliplatin | 0.6522 | 0.0257 | 0.6848 | 0.1963 | 0.9820 | 0.0422 |
| LP-void + | Oxaliplatin | 0.6060 | 0.0322 | 0.8987 | 0.2321 | 0.9976 | 0.0676 |
| Oxpt | | | | | | | |
| LP-Ox | Oxaliplatin | 0.8132 | 0.0432 | 0.8374 | 2.7621 | 0.9755 | 0.0726 |
| LP-Ox-AA | Oxaliplatin | 0.0006 | −0.0007 | 0.0614 | 0.0488 | 0.9777 | 0.0195 |
| LP-Stp | Satraplatin | 0.3561 | −0.0710 | 0.0535 | −0.1051 | 0.7060 | 0.0009 |
| LP-Stp + | Oxaliplatin | 0.7500 | 0.0404 | 0.9562 | 0.2693 | 0.9965 | 0.0790 |
| Oxpt | Satraplatin | 0.3654 | −0.0743 | 0.0594 | −0.1113 | 0.9965 | 0.6940 |
| LP-Ox-Stp | Oxaliplatin | 0.9589 | 0.0541 | 0.8897 | 0.2522 | 0.9745 | 0.0195 |
| | Satraplatin | 0.6576 | −0.0996 | 0.2996 | −0.2215 | 0.7265 | −0.0392 |

[symbol]

coefficient of determination, K [symbol] is the the zero-order release rate constant, K [symbol] is the firstorder release rate constant, K [symbol] is the Higuchi rate constant, K [symbol] is the cube root law release constant, n is the Korsmeyer peppas slope exponent, K [symbol] is the Baker-Lonsdale release rate constant, and K [symbol] is the Michaelis-Menten release rate constant. [symbol]
indicates data missing or illegible when filed

TABLE 11

| | IC₅₀ (μM) | | |
|---|---|---|---|
| Formulation | BHK-21 | HepG2 | MCF-7 |
| LP-Ox | 90.19 ± 2.053 | 52.05 ± 1.758 | 34.11 ± 1.504 |
| LP-Ox-AA | 74.81 ± 1.779 | 40.78* ± 1.504 | 31.84 ± 1.477 |
| LP-Ox-Stp | 58.45# ± 1.650 | 36.77** ± 1.568 | 34.55 ± 1.507 |
| Lipoxal | 53.44## ± 1.677 | 27.56** ± 1.575 | 28.75 ± 1.545 |
| Free | 72.06 ± 1.932 | 69.82 ± 1.631 | 50.37 ± 1.555 |
| Oxaliplatin | | | |

Data are means ± SD, n = 2.
*P < 0.05 difference from Free oxaliplatin.
**P < 0.01 difference from Free oxaliplatin.
#P < 0.05 difference from LP-Ox.
##P < 0.01 difference from LP-Ox.

What is claimed is:

1. A liposomal delivery composition for the treatment of cancer, comprising:
(a) a liposome composition comprising in a molar ratio:
distearoyl phosphatiylcholine (DSPC),
distearoyl phosphoethanolamine (DSPE),
distearoyl phosphatidylethanolamine-polyethylene glycol (DSPE-PEG), and
cholesterol;
(b) a first cancer drug encapsulated by the liposome composition; and
(c) a second cancer drug encapsulated by the liposome composition, where the first cancer drug is different from the second cancer drug.

2. The liposomal delivery composition as set forth in claim 1, wherein the first cancer drug is oxaliplatin.

3. The liposomal delivery composition as set forth in claim 1, wherein the liposomal delivery composition has negative surface potentials resulting in an encapsulation efficiency of about 20-25%

4. The liposomal delivery composition as set forth in claim 1, wherein the second cancer drug is a hydrophilic or hydrophobic.

5. The liposomal delivery composition as set forth in claim 1, wherein the second cancer drug is ascorbic acid.

6. The liposomal delivery composition as set forth in claim 1, wherein the second cancer drug is satraplatin.

7. The liposomal delivery composition as set forth in claim 1, wherein the molar ratio for DSPC in the composition ranges from 30-50%.

8. The liposomal delivery composition as set forth in claim 1, wherein the molar ratio for DSPE in the composition ranges from 3-5%.

9. The liposomal delivery composition as set forth in claim 1, wherein the molar ratio for DSPE-PEG in the composition ranges from 5-40%.

10. The liposomal delivery composition as set forth in claim 1, wherein the molar ratio for cholesterol in the composition ranges from 25-40%.

11. The liposomal delivery composition as set forth in claim 1, wherein the DSPE-PEG is phosphatidylethanolamine-polyethylene glycol 2000 (DSPE-PEG 2000).

12. The liposomal delivery composition as set forth in claim 1, wherein the liposomal delivery composition has a particle size of less than 200 nm.

13. The liposomal delivery composition as set forth in claim 1, wherein the liposomal delivery composition is to be administered through intravenous injection.

14. The liposomal delivery composition as set forth in claim 1, wherein the first drug is oxaplatin and the second drug is ascorbic acid with a molar ratio where the ascorbic acid is a fraction of about 0.01 to 0.05 higher than that of the oxaplatin.

15. The liposomal delivery composition as set forth in claim 1, wherein the first drug is oxaplatin and the second drug is satraplatin with a molar ratio where the satraplatin is about 5 times higher than than that of oxaplatin.
